(19) United States
(12) Patent Application Publication
(10) Pub. No.: US 2009/0076131 A1
(43) Pub. Date: Mar. 19, 2009

(54) Anti-Cancer and Anti-Inflammatory Effects of Annurca Apple Extracts and Compositions Purified Therefrom

(75) Inventors: Luigi Ricciardiello, Dallas, TX (US); Clement Richard Boland, Dallas, TX (US); Marco Romano, Napoli (IT); Vincenzo Fogliano, Rome (IT)

Correspondence Address: CHALKER FLORES, LLP, 2711 LBJ FRWY, Suite 1036, DALLAS, TX, 75234, US

(73) Assignee: BAYLOR RESEARCH INSTITUTE, Dallas, TX (US)

(21) Appl. No.: 12/182,266

(22) Filed: Jul. 30, 2008

Related U.S. Application Data
(60) Provisional application No. 60/952,751, filed on Jul. 30, 2007.

(51) Int. Cl. A61K 31/352; A61P 35/00; A61K 31/216

(52) U.S. Cl. 514/456; 514/533

(57) ABSTRACT

The present invention includes compositions and methods for modulating cell proliferation using a pharmaceutical effective amount of an isolated and purified polyphenolic composition having one or more polyphenolic compounds extracted from one or more plant tissues, specifically from one or more tissues of the Annurca apple.

### CROSS-REFERENCE TO RELATED APPLICATIONS

This application claims priority to U.S. Provisional Application Ser. No. 60/952,751, the entire contents of which are incorporated herein by reference.

### STATEMENT OF FEDERALLY FUNDED RESEARCH

This invention was made with U.S. Government support under Contract No. R01 CA72851 and R01 CA98572 awarded by the National Cancer Institute of the NIH. The government has certain rights in this invention.

### TECHNICAL FIELD OF THE INVENTION

The present invention relates in general to the field of cancer treatment and prevention, specifically methods for the preparation, use and formulations of polyphenols derived from apples for the treatment and/or prevention of cancer.

### BACKGROUND OF THE INVENTION

Without limiting the scope of the invention, its background is described in connection with methods for the preparation, use and formulations containing polyphenol derived from plants and fruits, specifically apples.

Colorectal cancer is one of the most common cancers in the United States and accounts for as many as one in ten cancer deaths. Colorectal cancer is dependent on many factors including family history, diet and other conditions. For example, diets high in fiber and low in fat show fewer instances of colorectal cancer at both the individual population as well as at the national level. However, high fiber-low fat diets are the opposite of the standard diets of the United States and many other developed countries.

Although colorectal cancer is treatable if discovered early, there are limited methods of detection available. The detection and removal of potentially cancerous polyps can greatly reduce the incidence and mortality rates of colorectal cancer. For example, the highly invasive and frequently uncomfortable endoscopic colonoscopy is a common technique employed to detect potentially cancerous polyps.

Along with early detection, prevention is a key factor in reducing the number of cancer deaths. Studies have shown a link between green tea and cancer prevention. Green tea has been used to provide antioxidant benefits including protection against the damage caused by smoke, pollution, stress and other toxins. In addition, green tea consumption was monitored in colorectal cancer patients and correlated with remission and/or slowing of growth. The polyphenolic compounds in green tea are thought to act as antioxidants to produce this effect; however, specific compounds were not identified or characterized.

Polyphenolic compounds, phenolic compounds or polyphenols are generally characterized by the presence of more than one phenol group or subcomponent per molecule and may be grouped by the type and number of phenolic and subcomponents present. In addition, the polyphenols can be generally subdivided further into tannins, and phenylpropanoids, e.g., lignans and flavonoids. Polyphenols are derived from numerous sources including fruit, fruit skin, plants, grains, nuts, berries, tea, beer, grapes, wine, olive oil, chocolate, cocoa, walnuts, peanuts, yerba mate, fruits and vegetables. As a result, the full extent (e.g., size, structure and members) of the polyphenolic compound family is undetermined. Even though more than 8,000 identified phenolic compounds have been identified the full extent of phenolic compounds is yet to be determined. To complicate further the identification and characterization of phenolic compounds, the polyphenolic compound family, the specific polyphenolic compounds present in individual genius, species and regional variations has yet to be characterized.

Studies have shown that anticancer properties of polyphenolic compounds vary in a compound specific manner with effects that include apoptosis, induction of cell cycle arrest, decrease in cell proliferation and modulation of epigenetic changes to name a few. However, the anticancer properties of polyphenolic compounds are compound dependent and cannot be classified into broad categories. The efficacy of polyphenolic compounds alone as a single therapy for the prevention, treatment or preventive of cancer in a patient is unknown. For example, green tea has been correlated with remission and/or slowing of growth of colorectal cancer in patients; however, the efficacy of epigallocatechin gallate (a polyphenolic compound present in green tea) as a single therapy is unknown.

As a result, compositions containing various combinations of polyphenolic compounds, extracts, and other therapeutic agents have been used to treat primary and metastatic cancers in humans. For example, U.S. Pat. No. 7,192,612 entitled “Compositions and methods based on synergies between capsicum extracts and tea catechins for prevention and treatment of cancer” provides methods and compositions of preventing or treating cancer by the administration of a combination of therapeutically effective amount of catechins, a group of polyphenols found in green tea and capsicum extracts. The compositions contain various combinations of the catechins and capsicum extracts, in combination with each other or other therapeutic agents and are used to treat primary and metastatic cancers in humans.

U.S. Pat. No. 6,096,359 entitled “Polyphenol fractions of tea, the use thereof and formulations containing them” provides the preparation of novel polyphenol fractions of camellia sinensis (tea), the use thereof and formulations containing them. The invention relates specifically to the preparation of extracts deprived of caffeine but containing the polyphenols deriving from epigallocatechin in a natural ratio. The use of these extracts, alone or in combination with other active principles, is of interest to the food, pharmaceutical, and cosmetic industry, especially to treat cytotoxic and oxidative conditions.

### SUMMARY OF THE INVENTION

The present inventors recognized that diets rich in fruits, vegetable, olive oil and red wine, were associated with a lower incidence of cancer due to the elevated amounts of phenolic compounds in those foods. The phenolic compounds have a broad spectrum of properties including antineoplastic, antioxidant and anti-inflammatory. The present inventors recognized that the Annurca apple of the Campania region in southern Italy is extremely rich in polyphenols that prevent exogenous damage in vitro to human gastric epithelial cells, and in vivo to rat gastric mucosa.

The present invention relates to methods for the preparation, use and as well as formulations containing polyphenol derived from plants and fruits for the treatment and/or prevention of cancer. The polyphenolic composition of the current invention is typically derived from plant or fruit tissues such as from an Annurca apple. One of many uses of the composition is for the treatment or prevention of colorectal cancer in a subject. The composition of the present invention may include a pharmaceutical carrier, and a pharmaceutical effective amount of polyphenolic compounds typically composed of catechins, chlorogenic acids, epicatechins or mixtures thereof. The polyphenolic composition may also be taken as a dietary supplement for the treatment or prevention of neoplasia in a subject.

The present invention provides methods for the treatment and/or prevention of cancer. In one embodiment, cell proliferation can be modulated by contacting cells with a pharmaceutical effective amount of a polyphenolic composition. In another embodiment, neoplasia can be treated in a subject by administering a pharmaceutical effective amount of a polyphenolic composition to the subject, where abnormal cell proliferation is affected. The proliferation of neoplasia cells may be modulated by inducing apoptosis and/or inducing expression of one or more tumor suppressor genes. The present invention also provides a method of effecting the DNA methylation of colorectal cancer cells, and a method of anticancer therapy by administering to a patient in need of anticancer therapy a pharmaceutical effective amount of a polyphenolic composition.

The present invention provides compositions and methods that reduce DNA methylation involving DNA methyl transferases (DNMTs), which catalyze the transfer of methyl groups to the carbon-5 position of cytosine in CpG islands. The present invention provides the inhibition of DNA methyl transferases. The lack of toxicity of compositions and extracts of the present invention make them excellent candidates for the chemoprevention.

The present invention includes treating neoplasia in a subject using a polyphenolic composition. The polyphenolic composition includes a pharmaceutical carrier and a pharmaceutical effective amount one or more polyphenolic compounds extracted from one or more tissues of an Annurca apple and affect one or more colorectal cancer cells, wherein the one or more polyphenolic compounds comprise catechins, chlorogenic acids, epicatechins or mixtures thereof.

The present invention includes a dietary supplement for the treatment or prevention of neoplasia in a subject. The polyphenolic composition includes a pharmaceutical carrier and a pharmaceutical effective amount one or more polyphenolic compounds extracted from one or more tissues of an Annurca apple and affect one or more colorectal cancer cells, wherein the one or more polyphenolic compounds comprise catechins, chlorogenic acids, epicatechins or mixtures thereof.

The present invention includes a method of inducing apoptosis using a polyphenolic composition. The polyphenolic composition includes a pharmaceutical carrier and a pharmaceutical effective amount one or more polyphenolic compounds extracted from one or more tissues of an Annurca apple and affect one or more colorectal cancer cells, wherein the one or more polyphenolic compounds comprise catechins, chlorogenic acids, epicatechins or mixtures thereof.

The present invention includes a method of effecting the DNA methylation of colorectal cancer cells using a polyphenolic composition. The polyphenolic composition includes a pharmaceutical carrier and a pharmaceutical effective amount one or more polyphenolic compounds extracted from one or more tissues of an Annurca apple and affect one or more colorectal cancer cells, wherein the one or more polyphenolic compounds comprise catechins, chlorogenic acids, epicatechins or mixtures thereof.

The present invention provides an increase in apoptosis, as seen in cell lines and showed no significant changes in cell cycle dynamics. Significant increases were detected in p21 and p53 in RKO cells after treatment (p<0.05). DNA methylation was significantly reduced in the promoters of hMLH 1, p14ARF and p16INK4a with consequent expression of the cognate RNAs and proteins. The effects of the present invention were comparable with those obtained with 5 aza-2 deoxycytidine (5-aza-2dC). A significant reduction in expression of DNMT proteins were observed after treatment without changes in mRNA. In addition, the present invention provides potent demethylating activity through the inhibition of DNMTs and lacks toxicity in Annurca extracts make them excellent candidates for the chemoprevention of colorectal cancer.

### BRIEF DESCRIPTION OF THE DRAWINGS

For a more complete understanding of the features and advantages of the present invention, reference is now made to the detailed description of the invention along with the accompanying figures and in which:

FIG. 1 is a graph of the percentage of cell viability after Annurca apple polyphenol extract treatment using the MTT assay;

FIG. 2A is a plot of the apoptotic response to Annurca apple polyphenol extract treatment evaluated by an annexinV/7AAD flow cytometry assay;

FIG. 2B is an image of a DNA fragmentation as a marker of late apoptosis evaluated by the TUNEL assay;

FIG. 3A is a plot of a flow cytometry analysis of the cell cycle distribution in Annurca apple polyphenol extract-treated samples and untreated controls;

FIG. 3B is an image of a Western blot of cell cycle regulatory proteins before and after Annurca apple polyphenol extract treatment;

FIG. 4 is an image that evaluates the methylation status of the hMLH1 promoter by methylation specific PCR;

FIG. 5A is a graph of the re-expression of hMLH1 transcripts by real time PCR in response to Annurca apple polyphenol extract treatment;

FIG. 5B is an image of a conventional RT-PCR amplification of hMLH1 in RKO after treatment with either 5-AZA-2dc or Annurca apple polyphenol extracts;

FIG. 5C is an image a western blot of hMLH1 protein expression;

FIGS. 6A-6B are images of gels illustrating the re-expression of previously silenced tumor suppressor genes by demethylation; and

FIGS. 7A and 7B are images of gels illustrating the expression of DNMT genes after Annurca apple polyphenol extract treatment in RKO cells.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

As used herein the term “carrier” is used to describe a substance, whether biodegradable or not, that is physiologically acceptable for human or animal use and may be pharmacologically active or inactive.

The term “immediate release” as used herein is used to describe a release profile to effect delivery of an active as soon as possible, that is, as soon as practically made available to an animal, whether in active form, as a precursor and/or as a metabolite. Immediate release may also be defined functionally as the release of over 80 to 90 percent (%) of the active ingredient within about 60, 90, 100 or 120 minutes or less. Immediate release as used herein may also be defined as making the active ingredient available to the patient or subject regardless of uptake, as some actives may never be absorbed by the animal. Immediate release formulations of the active on a carrier, such as rolled or compressed beads, may be formulated such that the surface area is maximized on beads and the active is exposed immediately. The immediate release formulations may also include effervescing agents that cause the disintegration of the structure integrity of the active and carrier such that release of the active is maximized. Various immediate release dosage forms may be designed readily by one of skill in art to achieve drug delivery to the stomach and small intestine, depending upon the choice of compression, adhesive materials and/or beading.

The terms “extended release” and “delayed release” as used herein is used to define a release profile to effect delivery of an active over an extended period of time, defined herein as being between about 60 minutes and about 2, 4, 6 or even 8 hours. Extended release may also be defined functionally as the release of over 80 to 90 percent (%) of the active ingredient after about 60 minutes and about 2, 4, 6 or even 8 hours. Extended release as used herein may also be defined as making the active ingredient available to the patient or subject regardless of uptake, as some actives may never be absorbed by the animal. Various extended release dosage forms may be designed readily by one of skill in art as disclosed herein to achieve delivery to both the small and large intestines, to only the small intestine, or to only the large intestine, depending upon the choice of coating materials and/or coating thickness.

“Extended release” and “delayed release” formulations may be prepared and delivered so that release is accomplished at some generally predictable location in the lower intestinal tract more distal to that which would have been accomplished if there had been no delayed release alterations. A method for delay of release is, e.g., a coating. Any coatings should be applied to a sufficient thickness such that the entire coating does not dissolve in the gastrointestinal fluids at pH below about 5, but does dissolve at pH about 5 and above. It is expected that any anionic polymer exhibiting a pH-dependent solubility profile can be used as an enteric coating in the practice of the present invention to achieve delivery to the lower gastrointestinal tract. Polymers and compatible mixtures thereof may be used to provide the coating for the delayed or the extended release of active ingredients, and some of their properties, include, but are not limited to: shellac, also called purified lac, a refined product obtained from the resinous secretion of an insect. This coating dissolves in media of pH>7.

The present pharmaceutical composition may also be provided in a variety of dosage forms, e.g., enveloped pharmaceutical, solution, suspension, cream, ointment, lotion, capsule, caplet, softgel, gelcap, elixir, syrup, emulsion, granule, gum, insert, jelly, paste, pastille, pellet, spray, lozenge, disk, magma, poultice, or wafer and the like. As used herein, the term “enveloped pharmaceutical” means a capsule, a suppository, a gel cap, a softgel, a lozenge, a sachet or even a fast dissolving wafer. The polyphenolic composition may be in the form of an immediate release, extended release or delayed release.

The term “modulate” as used herein, refers to a change or an alteration in the biological activity and may be an increase or a decrease in activity or any other change in the biological, functional, or immunological properties.

As used herein, the term “antioxidant” is intended to mean an agent which inhibits oxidation and thus is used to prevent the deterioration of preparations by the oxidative process. Such compounds include, by way of example and without limitation, ascorbic acid, ascorbyl palmitate, butylated hydroxyanisole, butylated hydroxytoluene, hypophosphorous acid, monothioglycerol, propyl gallate, sodium ascorbate, sodium bisulfite, sodium formaldehyde sulfoxylate and sodium metabisulfite and the like.

As used herein, the term “buffering agent” is intended to mean a compound used to resist change in pH upon dilution or addition of acid or alkali. Such compounds include, by way of example and without limitation, potassium metaphosphate, potassium phosphate, monobasic sodium acetate and sodium citrate anhydrous and dihydrate and the like.

As used herein, the term “colorant” is intended to mean a compound used to impart color to liquid and solid (e.g., tablets and capsules) pharmaceutical preparations. Such compounds include, by way of example and without limitation, FD&C Red No. 3, FD&C Red No. 20, FD&C Yellow No. 6, FD&C Blue No. 2, D&C Green No. 5, D&C Orange No. 5, D&C Red No. 8, caramel, and ferric oxide, red and the like.

As used herein, the term “flavorant” is intended to mean a compound used to impart a pleasant flavor and often odor to a pharmaceutical preparation. In addition to the natural flavorants, many synthetic flavorants are also used. Such compounds include, by way of example and without limitation, anise oil, cinnamon oil, cocoa, menthol, orange oil, peppermint oil and vanillin and the like.

As used herein, the term “sweetening agent” is intended to mean a compound used to impart sweetness to a preparation. Such compounds include, by way of example and without limitation, aspartame, dextrose, glycerin, mannitol, saccharin sodium, sorbitol and sucrose and the like.

As used herein, the term “tablet antiadherents” is intended to mean agents which prevent the sticking of table formulation ingredients to punches and dies in a tableting machine during production. Such compounds include, by way of example and without limitation, magnesium stearate, talc, and the like.

As used herein, the term “tablet binders” is intended to mean substances used to cause adhesion of powder particles in table granulations. Such compounds include, by way of example and without limitation, acacia, alginic acid, carboxymethyl cellulose, sodium, compressible sugar ethylcellulose, gelatin, liquid glucose, methylcellulose, povidone and pregelatinized starch and the like.

As used herein, the term “tablet and capsule diluent” is intended to mean inert substances used as fillers to create the desired bulk, flow properties, and compression characteristics in the preparation of tablets and capsules. Such compounds include, by way of example and without limitation, dibasic calcium phosphate, kaolin, lactose, mannitol, microcrystalline cellulose, powdered cellulose, precipitated calcium carbonate, sorbitol, and starch and the like.

As used herein, the term “tablet direct compression excipient” is intended to mean a compound used in direct compression tablet formulations. Such compounds include, by way of example and without limitation, dibasic calcium phosphate and the like.

As used herein, the term “tablet disintegrant” is intended to mean a compound used in solid dosage forms to promote the disruption of the solid mass into smaller particles, which are more readily dispersed or dissolved. Such compounds include, by way of example and without limitation, alginic acid, carboxymethylcellulose, calcium, microcrystalline cellulose, polacrilin potassium, sodium alginate, sodium starch glycolate, and starch and the like.

As used herein, the term “tablet glidant” is intended to mean agents used in tablet and capsule formulations to reduce friction during tablet compression. Such compounds include, by way of example and without limitation, colloidal silica, cornstarch, talc, and the like.

As used herein, the term “tablet lubricant” is intended to mean substances used in tablet formulations to reduce friction during tablet compression. Such compounds include, by way of example and without limitation, calcium stearate, magnesium stearate, mineral oil, stearic acid, zinc stearate, and the like.

As used herein, the term “tablet/capsule opaquant” is intended to mean a compound used to render a capsule or a tablet coating opaque. An opaquant may be used alone or in combination with a colorant. Such compounds include, by way of example and without limitation, titanium dioxide and the like.

As used herein, the term “tablet polishing agent” is intended to mean a compound used to impart an attractive sheen to coated tablets. Such compounds include, by way of example and without limitation, carnauba wax, white wax, and the like.

The term “pharmaceutically acceptable salts” refers to physiologically and pharmaceutically acceptable salts of the compounds of the invention: i.e., salts that retain the desired biological activity of the parent compound and do not impart undesired toxicological effects thereto.

Pharmaceutically acceptable base addition salts are formed with metals or amines, such as alkali and alkaline earth metals or organic amines. Examples of metals used as cations are sodium, potassium, magnesium, calcium, and the like. Examples of suitable amines are N,N′-dibenzylethylenediamine, chloroprocaine, choline, diethanolamine, dicyclohexylamine, ethylenediamine, N-methylglucamine, and procaine (see, for example, Berge et al., “Pharmaceutical Salts,” J. of Pharma Sci., 1977, 66, 119). The base addition salts of said acidic compounds are prepared by contacting the free acid form with a sufficient amount of the desired base to produce the salt in the conventional manner. The free acid form may be regenerated by contacting the salt form with an acid and isolating the free acid in the conventional manner. The free acid forms differ from their respective salt forms somewhat in certain physical properties such as solubility in polar solvents, but otherwise the salts are equivalent to their respective free acid for purposes of the present invention. As used herein, a “pharmaceutical addition salt” includes a pharmaceutically acceptable salt of an acid form of one of the components of the compositions of the invention. These include organic or inorganic acid salts of the amines. Preferred acid salts are the hydrochlorides, acetates, salicylates, nitrates and phosphates. Other suitable pharmaceutically acceptable salts are well known to those skilled in the art and include basic salts of a variety of inorganic and organic acids, such as, for example, with inorganic acids, such as for example hydrochloric acid, hydrobromic acid, sulfuric acid or phosphoric acid; with organic carboxylic, sulfonic, sulfo or phospho acids or N-substituted sulfamic acids, for example acetic acid, propionic acid, glycolic acid, succinic acid, maleic acid, hydroxymaleic acid, methylmaleic acid, fumaric acid, malic acid, tartaric acid, lactic acid, oxalic acid, gluconic acid, glucaric acid, glucuronic acid, citric acid, benzoic acid, cinnamic acid, mandelic acid, salicylic acid, 4-aminosalicylic acid, 2-phenoxybenzoic acid, 2-acetoxybenzoic acid, embonic acid, nicotinic acid or isonicotinic acid; and with amino acids, such as the 20 alpha-amino acids involved in the synthesis of proteins in nature, for example glutamic acid or aspartic acid, and also with phenylacetic acid, methanesulfonic acid, ethanesulfonic acid, 2-hydroxyethanesulfonic acid, ethane-1,2-disulfonic acid, benzenesulfonic acid, 4-methylbenzenesulfonic acid, naphthalene-2-sulfonic acid, naphthalene-1,5-disulfonic acid, 2- or 3-phosphoglycerate, glucose-6-phosphate, N-cyclohexylsulfamic acid (with the formation of cyclamates), or with other acid organic compounds, such as ascorbic acid. Pharmaceutically acceptable salts of compounds may also be prepared with a pharmaceutically acceptable cation. Suitable pharmaceutically acceptable cations are well known to those skilled in the art and include alkaline, alkaline earth, ammonium and quaternary ammonium cations. Carbonates or hydrogen carbonates are also possible.

As used herein, the terms “Polyphenolic compounds,” “Phenolic compounds,” “Polyphenols” and “Flavanols” may be used interchangeably to characterized compounds having one or more phenol groups or subcomponent per molecule. Within the general term “polyphenols” are also included the dihydroxy and trihydroxy benzoic acids and the phytoalexins. Other examples include catechins flavonoids, polyphenols, Procyanidin polyphenols, flavones, flavanols, flavan-3-ols, proanthocyanidins, procyanidols, procyanins, procyanidins, and tannins. The term is also intended to encompass associated derivatives which are additionally substituted, especially those compounds which include substitutions and modification including but not limited to alkylation, methylation, ethylation, alkoxyation, sulphation, malonylation and one or more sugar residues (such as glucose, galactose, arabinose, rhamnose and the like), particularly O-glycosylated compounds so forth. The compounds also include compounds that may be classified into tannins, and phenylpropanoids, e.g., lignins and flavonoids. The compounds may be derived from numerous sources including fruit, fruit skin, plants, grains, nuts, berries, tea, beer, grapes, wine, olive oil, chocolate, cocoa, walnuts, peanuts, yerba mate, fruits and vegetables.

As used herein, the terms “Cancer” means an increase in the number of abnormal cells derived from a given normal tissue or any clinical definition. In addition, it may involve the invasion of adjacent or non-adjacent tissues and/or the lymphatic or blood-borne spread of malignant cells to other sites and/or regional lymph nodes. Furthermore, the term also encompasses hyperplasia, precancerous cells and minor preneoplastic changes.

As used herein, the term “Preventing cancer” means to inhibit the transformation of a cell into an abnormal cell by a carcinogenic agent or agents and/or to inhibit the accumulation of cells expressing cancer-specific genes to a number that creates one or more clinical symptoms associated with cancer.

As used herein, the terms “Treating cancer” and “treatment of cancer” mean to at least partially inhibit the replication of cancer cells, to inhibit the spread of cancer, to decrease tumor size, to lessen or reduce the number of cancerous cells in the body, and to ameliorate or alleviate the symptoms of the disease caused by the cancer. The treatment is considered therapeutic if there is a decrease in mortality and/or morbidity.

As used herein, “Extraction” refers to a technique for separating a mixture of chemical components, wherein the components that are separated have different solubilities, molecular weights, charge, adsorption strengths, ionic strengths or a combination thereof. In some instances, the components separated from a mixture of compounds from plant tissue. A “Solvent extraction” is a type of extraction wherein a mixture of components are separated utilizing the differences in the solubilities and adsorption strengths of the components that are separated. The skilled artisan will recognize other methods for extracting and separating the compounds of the present invention, see e.g., U.S. Pat. No. 7,198,808, which provides a method for selectively extracting acidic and/or non-acidic compounds from natural material such as plant tissue and is incorporated by reference in its entirety.

The present invention relates to methods for the preparation, use and as well as formulations containing polyphenol derived from plants and fruits for the treatment and/or prevention of cancer. The polyphenol composition of the present invention typically has one or more polyphenolic compounds extracted from plant tissues, and usually contain catechins, chlorogenic acids, epicatechins or mixtures thereof. The concentration of the composition is typically between 1 mM and 20 mM of each catechins, chlorogenic acid and epicatechin, but may be different depending on the application. The polyphenolic composition may be derived from multiple different sources. Examples include, but not limited to apples, such as Annurca apples, blackberries, blueberries, cantaloupe, cherries, cranberries, grapes, pears, plums, raspberries, strawberries, broccoli, cabbage, celery, onion parsley, red wine, chocolate, green tea, olive oil, bee pollen and grains or mixtures thereof.

The present invention provides a method of modulating cell proliferation by contacting cells with a pharmaceutical effective amount of the polyphenolic composition. Cell proliferation may be modulated in many ways, some examples include, but not limited to modifying apoptosis, inducting cell cycle arrest, decreasing cell proliferation, modulating epigenetic changes or a combinations thereof.

Generally, the skilled artisan will recognize the structure of polyphenol as used herein, e.g., the flavones are compounds with two benzene rings linked with a heterocyclic six member ring C containing a carbonyl group. For example, one benzene ring can be joined in position 2 to give a flavone or to position 3 to give an iso-flavone. Hydroxylation can occur at one or more of positions 3, 5, 7, and 3′, 4′, 5′ to give compounds. Typical examples of flavonols are: quercetin (e.g., hydroxylated at positions 3, 5, 7, 3′, 4′), kaempferol (e.g., hydroxylated at positions 3, 5, 7, 4′), and myricetin (e.g., hydroxylated at positions 3, 5, 7, 3′, 4′, 5′). The two most common flavanols or polyphenols are catechin (e.g., hydroxyl groups at positions 5, 7, 3′, 4′) and its stereo-isomer epi-catechin. The hydroxyl groups can be esterified with gallic acid. The proanthocyanidins are polymers of catechin and/or epicatechin and can contain up to 8 or more repeat units. These compounds are often called proanthocyanidins, procyanidins or tannins.

The polyphenol compositions of the present invention also describe a class of substituted phenolic compounds that are also known as flavanols or catechins. The polyphenols include apigenin, catechin, epicatechin, gallocatechin, gallocatechin gallate, epigallocatechin, epicatechin gallate, epigallocatechin gallate, anthocyanins, caffeine, theobromine, theophylline, phenolic acid, quercetin, ellagic acid, nobotanin and gallic acid.

In addition, the present invention also includes polyphenol that are modified and/or substituted, e.g., aglycone or O-glycosides (e.g. with D-glucose, galactose, arabinose, rhamnose etc). The term polyphenols and flavanols is also intended to encompass associated derivatives which are additionally substituted, especially those compounds which include substitutions and modification including but not limited to alkylation, methylation, ethylation, alkoxyation, sulphation, malonylation and one or more sugar residues (such as glucose, galactose, arabinose, rhamnose and the like), particularly O-glycosylated compounds so forth.

In addition the polyphenolic composition of the present invention may be coated. For example, coated particle for use with the present invention is disclosed in U.S. Pat. No. 4,221,778, in which a selective, prolonged continuous release of pharmacologically active drugs, under conditions such as those encountered in the gastrointestinal tract, is achieved by the application of a diffusion barrier coating to an ion exchange drug-resin complex particle which has been treated with a solvating agent. Another prolonged release formulations from coated drugs may be prepared under circumstances wherein a component of the formulation includes a second ionic substance (e.g., a combination drug, a dye, a dispersing agent or the like) bearing the same ionic charge as the drug on the drug-resin complex by employing the second ionic substance in the ion form of an exchange resin complex. The manufacture of a formulation of any drug for liquid dosage usage requires that the final formulation have the drug dissolved or suspended in a liquid that possess extended shelf-life stability and exhibit no change in active drug dosage level over a period of time and has acceptable taste. Thus, to prepare a liquid formulation of any type drug it may be necessary to employ extenders such as water or syrup, and to add flavors, sweeteners, thickening agents, dyes, and the like. To control the dissolution profile of the formulation versus the dissolution profile of the same drug in water, the coated particles may also be included in the presence of ionic substances bearing the same ionic charge as the sustained release drug present in the formulation as a coated drug-resin complex. The presence of ionic substances of opposite charge in the final solution, do not have an effect on the expected dissolution rate and improve the release profile. In fact, the second ionic material need not be coated with the water-permeable diffusion barrier coating.

Wurster coating. Examples of resin drug complexes for rapid release of a drug in 0.1 normal hydrochloric acid (0.1N HCl) dissolution medium (which simulates the fluids of the gastrointestinal tract) include, e.g., an uncoated and untreated Amberlite IRP-69 phenylpropanolamine complex with a 22.5% drug loading released 86.3% of the drug in 1 hour. Some retardation of this rapid release can be obtained by attempting to coat the complex particles, without glycerin pretreatment, with a diffusion barrier coating. The efficiency of the coating on the complex particles can be improved and the release of the drug further slowed by treating the resin particles prior to coating with, e.g., about 15-25% glycerin, resulting in the ability to selectively prolong the release of drugs from drug-resin complexes. While the glycerin may be applied to the drug-resin complex, it may be applied to the resin prior to complexing, as in the case where the resin particles are coated prior to complexing with the drug.

Compositions and formulations for parenteral, intrathecal or intraventricular administration may include sterile aqueous solutions, which may also contain buffers, diluents and other suitable additives such as, but not limited to, penetration enhancers, carrier compounds and other pharmaceutically acceptable carriers or excipients.

The pharmaceutical formulations of the present invention, which may conveniently be presented in unit dosage form, may be prepared according to conventional techniques well known in the pharmaceutical industry. Such techniques include the step of bringing into association the active ingredients with the pharmaceutical carrier(s) or excipient(s). In general the formulations are prepared by uniformly and intimately bringing into association the active ingredients with liquid carriers or finely divided solid carriers or both, and then, if necessary, shaping the product.

Pharmaceutical compositions of the present invention include, but are not limited to, solutions, emulsions, and liposome-containing formulations. These compositions may be generated from a variety of components that include, but are not limited to, preformed liquids, self-emulsifying solids and self-emulsifying semisolids. For oral, buccal, and sublingual administration, the pharmaceutical composition of the invention may be administered as either solutions or suspensions in the form of gelcaps, caplets, tablets, capsules or powders.

For gelcap preparations, the pharmaceutical formulation may include oils, e.g.: (1) fixed oils, such as peanut oil, sesame oil, cottonseed oil, corn oil and olive oil; (2) fatty acids, such as oleic acid, stearic acid and isostearic acid; and fatty acid esters, such as ethyl oleate, isopropyl myristate, fatty acid glycerides and acetylated fatty acid glycerides; (3) alcohols, such as ethanol, isopropanol, hexadecyl alcohol, glycerol and propylene glycol; (4) glycerol ketals, such as 2,2-dimethyl-1,3-dioxolane-4-methanol; (5) ethers, such as poly(ethylene glycol) 450; (6) petroleum hydrocarbons, such as mineral oil and petrolatum; and (7) water, or with mixtures thereof, with or without the addition of a pharmaceutically suitable surfactant, suspending agent or emulsifying agent.

Adsorption of the complexes or compounds onto the ion exchange resin particles to form the active agent-resin complex is a well-known technique as shown in U.S. Pat. No. 2,990,332 (relevant portions incorporated herein by reference) and demonstrated in the examples herein. In general, the drug is mixed with an aqueous suspension of the resin and the complex is then dried. Adsorption of drug onto the resin is detected by a change in the pH of the reaction medium.

Liquid Dosage Forms: The liquid dosage formulation and method of this invention may be applied generally to liquid formulations of active agents, which may be prepared conventionally as described herein and, for example, to those liquid formulations contained in commercially-available dosage forms that include the two or more salts of the same active agent. The liquid may be provided as an encapsulated liquid formulation.

The dosage form may also include an antioxidant to slow or effectively stop the rate of any auto-oxidizable material present in the dosage form, particularly if it is in a liquid formulation within, e.g., a gelatin capsule. Representative antioxidants include, e.g., ascorbic acid; alpha tocopherol; ascorbyl palmitate; ascorbates; isoascorbates; butylated hydroxyanisole; butylated hydroxytoluene; nordihydroguaiaretic acid; esters of garlic acid having at least 3 carbon atoms comprising a member selected from the group consisting of propyl gallate, octyl gallate, decyl gallate, decyl gallate; 6-ethoxy-2,2,4-trimethyl-1,2-dihydro-quinoline; N-acetyl-2,6-di-t-butyl-p-aminophenol; butyl tyrosine; 3-tertiarybutyl-4-hydroxyanisole; 2-tertiary-butyl-4-hydroxyanisole; 4-chloro-2,6-ditertiary butyl phenol; 2,6-ditertiary butyl p-methoxy phenol; 2,6-ditertiary butyl-p-cresol: polymeric antioxidants; trihydroxybutyro-phenone physiologically acceptable salts of ascorbic acid, erythorbic acid, and ascorbyl acetate; calcium ascorbate; sodium ascorbate; sodium bisulfite; and the like. The amount of antioxidant used for the present purposes may be about 0.001% to 25% of the total weight of the composition present in the dosage form. Antioxidants are known to the prior art in U.S. Pat. Nos. 2,707,154; 3,573,936; 3,637,772; 4,038,434; 4,186,465 and 4,559,237, relevant portions incorporated herein by reference.

The liquid dosage form may also contain one or more chelating agents to protect the active agent either during storage or when in use. Examples of chelating agents include polyacrylic acid, citric acid, edetic acid, disodium edetic acid, and the like. The chelating agent may be co-delivered with the active agent in the environment of use to preserve and protect the active agent in situ. Such chelating agents may be combined with the liquid, active agent formulation in the porous particles, or the chelating agents may be incorporated into the drug layer in which the porous particles are dispersed.

The liquid formulation may also include one or more surfactants, e.g., nonionic, anionic and cationic surfactants, or combinations thereof. Examples of nontoxic, nonionic surfactants suitable for forming a liquid-based formulation include, e.g., alkylated aryl polyether alcohols; polysorbates such as polysorbate 80; polyethylene glycol tertdodecyl throether available as; fatty and amide condensate or; aromatic polyglycol ether condensate; fatty acid alkanolamine or sorbitan monolaurate; polyoxyethylene sorbitan esters; sorbitan monolaurate polyoxyethylene; sorbitan mono-oleate polyoxyethylene; polyoxypropylene-polyoxyethylene; polyglycolyzed glycerides such as Labraosol, polyoxyethylated castor oil such as Cremophor and polyoxypropylene-polyoxyethylene-8500. By way of example, anionic surfactants include, e.g., sulfonic acids and the salts of sulfonated esters such as sodium lauryl sulfate, sodium sulfoethyl oleate, dioctyl sodium sulfosuccinate, cetyl sulfate sodium, myristyl sulfate sodium; sulfated esters; sulfated amides; sulfated alcohols; sulfated ethers; sulfated carboxylic acids; sulfonated aromatic hydrocarbons; sulfonated ethers; and the like. Cationic surface active agents for use with liquid formulations, include, e.g., cetyl pyridinium chloride; cetyl trimethyl ammonium bromide; diethylmethyl cetyl ammonium chloride; benzalkonium chloride; benzethonium chloride; primary alkyl ammonium salts; secondary alkyl ammonium salts; tertiary alkyl ammonium salts; quaternary alkyl ammonium salts; acylated polyamines; salts of heterocyclic amines; palmitoyl carnitine chloride, behentrimonium methosulfate, and the like. Surfactants with be provided generally, from 0.01 part to 1000 parts by weight of surfactant, per 100 parts of the active agent.

It is contemplated that the present invention may be formulated as an “immediate release” and/or an “extended release” or “delayed release”, e.g., freeze dried, rotary dried or spray dried powders; amorphous or crystalline powders; granules, precipitates or particulates. The immediate release active may be either free-flowing or compressed.

The pharmaceutical formulation may further include, e.g., water, aqueous solvents, non-protic solvents, protic solvents, hydrophilic solvents, hydrophobic solvents, polar solvents, non-polar solvent, emollients and/or combinations thereof. Other formulations may include, optionally, stabilizers, pH modifiers, surfactants, perfumes, astringents, cosmetic foundations, pigments, dyes, bioavailability modifiers and/or combinations thereof.

Effervescent pharmaceutical formulations are well known in the art and include, generally, an acid such as citric acid or a mono or dihydrogen salt thereof and a carbon dioxide source such as a carbonate or hydrogen carbonate alkali metal salt, such as sodium hydrogen carbonate. The acid and the carbon dioxide source do not react together when dry but combine to release carbon dioxide and an effervescent effect in the presence of water. The effervescent pharmaceutical compositions for use with the present invention may be in the form of a tablet for dissolving in water or a dispersible powder for sprinkling onto water, prior to administration. The acid and the carbon dioxide source are blended together during manufacture of the composition in the absence of water to prevent premature effervescence.

Effervescent pharmaceutical compositions may be in the form of a tablet for dissolving in water or a dispersible powder for sprinkling onto water, prior to administration. The components of the couple are blended together during manufacture of the composition. Suitable pharmaceutical formulations include effervescent tablets and sachets containing water dispersible powders. Effervescent pharmaceutical formulations according to the present invention may be prepared by blending together granulates formed by roller compaction with other components prior to processing into, e.g., beads. Roller compaction may also be extended to include other components, such as one or more active ingredients and non-active ingredients or excipients such as lubricants, disintegrants, flavors and sweeteners. For capsule, final processing may include introducing the beads into the capsules using an encapsulation machine.

Monosodium citrate and sodium bicarbonate are blended together and then roller compacted, in the absence of water, to form flakes that are then crushed to give granulates. The granulates are then combined with the active ingredient or drug or salt thereof, conventional beading or filling agents and, optionally, sweeteners, flavors and lubricants. The granules are then filled together under controlled ambient conditions, to form beads or capsules, respectively. The hardness of the final tablets is influenced by the linear roller compaction strength used in preparing granulates, which are influenced by the particle size of the monosodium hydrogen carbonate and sodium hydrogen carbonate.

Other additives conventionally used in pharmaceutical compositions may be included, which are well known in the art. Such additives include, anti-adherents, anti-sticking agents, glidants, flow promoters, lubricants, talc, magnesium stearate, fumed silica, micronized silica, polyethylene glycols, surfactants, waxes, stearic acid, stearic acid salts, stearic acid derivatives, starch, hydrogenated vegetable oils, sodium benzoate, sodium acetate, leucine, PEG-4000 and magnesium lauryl sulfate.

Other additives include, binders (adhesives), i.e., agents that impart cohesive properties to powdered materials through particle-particle bonding, such as matrix binders (e.g., dry starch, dry sugars), film binders (e.g., PVP, starch paste, celluloses, bentonite and sucrose), and chemical binders (e.g., polymeric cellulose derivatives, e.g., carboxy methyl cellulose, HPC and HPMC; sugar syrups; corn syrup; water soluble polysaccharides such as acacia, tragacanth, guar and alginates; gelatin; gelatin hydrolysate; agar; sucrose; dextrose; and non-cellulosic binders, such as PVP, PEG, vinyl pyrrolidone copolymers, pregelatinized starch, sorbitol, and glucose.

For certain actives it may be useful to provide buffering agents (or bufferants), where the acid is a pharmaceutically acceptable acid, such as hydrochloric acid, hydrobromic acid, hydriodic acid, sulfuric acid, nitric acid, boric acid, phosphoric acid, acetic acid, acrylic acid, adipic acid, alginic acid, alkanesulfonic acid, amino acids, ascorbic acid, benzoic acid, boric acid, butyric acid, carbonic acid, citric acid, fatty acids, formic acid, fumaric acid, gluconic acid, hydroquinosulfonic acid, isoascorbic acid, lactic acid, maleic acid, methanesulfonic acid, oxalic acid, para-bromophenylsulfonic acid, propionic acid, p-toluenesulfonic acid, salicylic acid, stearic acid, succinic acid, tannic acid, tartaric acid, thioglycolic acid, toluenesulfonic acid and uric acid, and where the base is a pharmaceutically acceptable base, such as an amino acid, an amino acid ester, ammonium hydroxide, potassium hydroxide, sodium hydroxide, sodium hydrogen carbonate, aluminum hydroxide, calcium carbonate, magnesium hydroxide, magnesium aluminum silicate, synthetic aluminum silicate, synthetic hydrotalcite, magnesium aluminum hydroxide, diisopropylethylamine, ethanolamine, ethylenediamine, triethanolamine, triethylamine, triisopropanolamine, or a salt of a pharmaceutically acceptable cation and acetic acid, acrylic acid, adipic acid, alginic acid, alkanesulfonic acid, an amino acid, ascorbic acid, benzoic acid, boric acid, butyric acid, carbonic acid, citric acid, a fatty acid, formic acid, fumaric acid, gluconic acid, hydroquinosulfonic acid, isoascorbic acid, lactic acid, maleic acid, methanesulfonic acid, oxalic acid, para-bromophenylsulfonic acid, propionic acid, p-toluenesulfonic acid, salicylic acid, stearic acid, succinic acid, tannic acid, tartaric acid, thioglycolic acid, toluenesulfonic acid, and uric acid.

As used herein, the term “Gastrointestinal inflammation” refers to inflammation of a mucosal layer of the gastrointestinal tract, and encompasses acute and chronic inflammatory conditions. Acute inflammation is generally characterized by a short time of onset and infiltration or influx of neutrophils. Chronic inflammation is generally characterized by a relatively longer period of onset and infiltration or influx of mononuclear cells. Chronic inflammation can also typically characterized by periods of spontaneous remission and spontaneous occurrence. “Mucosal layer of the gastrointestinal tract” is meant to include mucosa of the bowel including the small intestine and large intestine, rectum, stomach (gastric) lining, oral cavity, and the like.

As used herein, the term “Chronic gastrointestinal inflammation” refers to inflammation of the mucosal of the gastrointestinal tract that is characterized by a relatively longer period of onset, is long-lasting (e.g., from several days, weeks, months, or years and up to the life of the subject), and is associated with infiltration or influx of mononuclear cells and can be further associated with periods of spontaneous remission and spontaneous occurrence. Thus, subjects with chronic gastrointestinal inflammation may be expected to require a long period of supervision, observation, or care. “Chronic gastrointestinal inflammatory inflammation” is also referred to as “chronic gastrointestinal inflammatory diseases” or “chronic gastrointestinal inflammatory conditions”. Chronic gastrointestinal inflammation may include, but are not limited to, inflammatory bowel disease, colitis induced by environmental insults such as administration of chemotherapy, radiation therapy and the like, colitis in conditions such as chronic granulomatous disease (Schappi et al. Arch Dis Child. 2001 February; 1984(2):147-151), celiac disease, celiac sprue, food allergies, gastritis, infectious gastritis or enterocolitis, and other forms of gastrointestinal inflammation caused by an infectious agent, and other similar conditions.

As used herein, “Inflammatory bowel disease” or “IBD” refers to any of a variety of diseases characterized by inflammation of all or part of the intestines. Examples of inflammatory bowel disease include, but are not limited to, Crohn's disease and ulcerative colitis.

In certain embodiments, the present invention may be used for treatment and/or prevention of gastrointestinal inflammation such as inflammatory bowel disease. The term “Inflammatory Bowel Disease” is commonly used to refer to a group of related, but distinct, chronic inflammatory conditions affecting the gastrointestinal tract. Abnormal p53 expression is a key early step in colon carcinogenesis rising in the setting of chronic inflammation, and abnormal p53 regulation can be found in chronically inflamed tissues. Inflammatory bowel disease may be Crohn's disease (CD) and ulcerative colitis (UC), both of which are idiopathic chronic diseases occurring with an increasing frequency in many parts of the world. In the United States, more than 600,000 are affected every year. IBD can involve either or both small and large bowel. CD can involve any part of the gastrointestinal tract, but most frequently involves the distal small bowel and colon. It either spares the rectum, or causes inflammation or infection with drainage around the rectum. UC usually causes ulcers in the lower part of the large intestine, often starting at the rectum. Symptoms vary but may include diarrhea, fever, and pain. Patients with prolonged UC are at an increased risk of developing colorectal cancer. Inflammatory bowel disease may also include other disease such as non-ulcerative colitis, carcinomas, polyps, cysts of the colon and/or rectum, or combinations thereof.

In another embodiment, the present invention may be used to reduce, prevent, and/or manage inflammatory bowel disease, related gastrointestinal pathologies, and symptoms thereof. The inflammatory bowel disease may be associated with one or more intestinal conditions. Thus, in certain embodiments, the present invention may also be used to directly or indirectly reduce, prevent, and/or manage intestinal conditions. Examples of intestinal conditions may include, but are not limited to, inflammatory bowel disease, ulcerative colitis, indeterminate colitis, infectious colitis, granulomatous enteritis, Crohn's disease, irritable bowel syndrome, infectious diseases of the small and large intestine, pyloric spasm, abdominal cramps, functional gastrointestinal disorders, mild dysenteries, diverticulitis, acute enterocolitis, neurogenic bowel disorders, including the splenic flexure syndrome and neurogenic colon, spastic colitis, cysts, polyps, and carcinoma.

The compositions of the present invention may additionally contain other adjunct components conventionally found in pharmaceutical compositions, at their art-established usage levels. Thus, for example, the compositions may contain additional, compatible, pharmaceutically-active materials such as, for example, antipruritics, astringents, local anesthetics or anti-inflammatory agents, or may contain additional materials useful in physically formulating various dosage forms of the compositions of the present invention, such as dyes, flavoring agents, preservatives, antioxidants, opacifiers, thickening agents and stabilizers. However, such materials, when added, should not unduly interfere with the biological activities of the components of the compositions of the present invention. The formulations can be sterilized and, if desired, mixed with auxiliary agents, e.g., lubricants, preservatives, stabilizers, wetting agents, emulsifiers, salts for influencing osmotic pressure, buffers, colorings, flavorings and/or aromatic substances and the like which do not deleteriously interact with the formulation.

Certain embodiments of the invention provide pharmaceutical compositions containing (a) one or more antisense compounds and (b) one or more other chemotherapeutic agents which function by a non-antisense mechanism. Examples of such chemotherapeutic agents include but are not limited to daunorubicin, daunomycin, dactinomycin, doxorubicin, epirubicin, idarubicin, esorubicin, bleomycin, mafosfamide, ifosfamide, cytosine arabinoside, bis-chloroethylnitrosurea, busulfan, mitomycin C, actinomycin D, mithramycin, prednisone, hydroxyprogesterone, testosterone, tamoxifen, dacarbazine, procarbazine, hexamethylmelamine, pentamethylmelamine, mitoxantrone, amsacrine, chlorambucil, methylcyclohexylnitrosurea, nitrogen mustards, melphalan, cyclophosphamide, 6-mercaptopurine, 6-thioguanine, cytarabine, 5-azacytidine, hydroxyurea, deoxycoformycin, 4-hydroxyperoxycyclophosphoramide, 5-fluorouracil (5-FU), 5-fluorodeoxyuridine (5-FUdR), methotrexate (MTX), colchicine, taxol, vincristine, vinblastine, etoposide (VP-16), trimetrexate, irinotecan, topotecan, gemcitabine, teniposide, cisplatin and diethylstilbestrol (DES). See, generally, The Merck Manual of Diagnosis and Therapy, 15th Ed. 1987, pp. 1206 1228, Berkow et al., eds., Rahway, N.J. When used with the compounds of the invention, such chemotherapeutic agents may be used individually (e.g., 5-FU and oligonucleotide), sequentially (e.g., 5-FU and oligonucleotide for a period of time followed by MTX and oligonucleotide), or in combination with one or more other such chemotherapeutic agents (e.g., 5-FU, MTX and oligonucleotide, or 5-FU, radiotherapy and oligonucleotide). Anti-inflammatory drugs, including but not limited to nonsteroidal anti-inflammatory drugs and corticosteroids, and antiviral drugs, including but not limited to ribivirin, vidarabine, acyclovir and ganciclovir, may also be combined in compositions of the invention. See, generally, The Merck Manual of Diagnosis and Therapy, 15th Ed., Berkow et al., eds., 1987, Rahway, N.J., pages 2499 2506 and 46 49, respectively). Other non-antisense chemotherapeutic agents are also within the scope of this invention. Two or more combined compounds may be used together or sequentially.

It is contemplated that the “immediate release” active may be formulated as, e.g., freeze dried, rotary dried or spray dried powders; amorphous or crystalline powders; granules, precipitates or particulates. The immediate release active may be either free-flowing or compressed. The pharmaceutical formulation may further include, e.g., water, aqueous solvents, non-protic solvents, protic solvents, hydrophilic solvents, hydrophobic solvents, polar solvents, non-polar solvent, emollients and/or combinations thereof. Other formulations may include, optionally, stabilizers, pH modifiers, surfactants, perfumes, astringents, cosmetic foundations, pigments, dyes, bioavailability modifiers and/or combinations thereof.

The present invention provides compositions and methods that reduce DNA methylation involving DNA methyl transferases (DNMTs), which catalyze the transfer of methyl groups to the carbon-5 position of cytosines in CpG islands. The present invention provides the inhibition of DNA methyl transferases. The present invention may modulate one or more tumor suppression genes and in turn modulate the growth of one or more colon carcinoma cells, colorectal cancer cells, rectal carcinoma cells, hairy cell leukemia cells, osophogeal carcinoma cells, sarcoma cells, seminoma cells, angiosarcoma cells, carcinoma cells, chordoma cells, fibrosarcoma cells, myxosarcoma cells, liposarcoma cells, chondrosarcoma cells, osteogenic sarcoma cells, endotheliosarcoma cells, lymphangiosarcoma cells, lymphangioendotheliosarcoma cells, synovioma cells, mesothelioma cells, leiomyosarcoma cells, rhabdomyosarcoma cells, pancreatic cancer cells, breast cancer cells, ovarian cancer cells, prostate cancer cells, squamous cell carcinoma cells, basal cell carcinoma cells, adenocarcinoma cells, sweat gland carcinoma cells, sebaceous gland carcinoma cells, papillary carcinoma cells, papillary adenocarcinomas cells, cystadenocarcinoma cells, medullary carcinoma cells, bronchogenic carcinoma cells, renal cell carcinoma cells, hepatoma cells, bile duct carcinoma cells, choriocarcinoma cells, embryonal carcinoma cells, cervical cancer cells, testicular tumor cells, lung carcinoma cells, small cell lung carcinoma cells, bladder carcinoma cells, epithelial hemangioblastoma cells, acoustic neuroma cells, oligodendroglioma cells, meningioma cells, melanoma cells, neuroblastoma cells, retinoblastoma cells, acute lymphocytic leukemia cells, acute myelocytic leukemia cells, promyelocytic leukemia cells, myelomonocytic leukemia cells, monocytic leukemia cells, erythroleukemia leukemia cells, chronic myelocytic leukemia cells, chronic lymphocytic leukemia cells, polycythemia vera cells, lymphoma cells, hodgkin's disease cells, non-hodgkin's disease cells, multiple myeloma cells, waldenstrom's macroglobulinemia cells, ewing's tumor cells, wilms' tumor cells and combinations thereof.

In a one embodiment, the patient may not have cancer, may be undergoing treatment for cancer, or may already have cancer, have cancer but no metastasis, have cancer and a metastatic cancer, have cancer that is in remission, have cancer that is immunosuppressed as a result of undergone anti-cancer therapy, chemotherapy, radiation or a combination thereof prior to administration of the invention.

The present pharmaceutical composition may also be provided in a variety of dosage forms, (e.g., enveloped pharmaceutical, solution, suspension, cream, ointment, lotion, capsule, caplet, softgel, gelcap, elixir, syrup, emulsion, granule, gum, insert, jelly, paste, pastille, pellet, spray, lozenge, disk, magma, poultice, or wafer) and the like and may contain various additives, e.g.: anti-adherents, anti-sticking agents, glidants, flow promoters, lubricants, talc, magnesium stearate, fumed silica, micronized silica, polyethylene glycols, surfactants, waxes, stearic acid, stearic acid salts, stearic acid derivatives, starch, hydrogenated vegetable oils, sodium benzoate, sodium acetate, leucine, magnesium lauryl sulfate, carrier, anti-adherents, anti-sticking agents, glidants, flow promoters, lubricants, talc, magnesium stearate, fumed silica, micronized silica, polyethylene glycols, surfactants, waxes, stearic acid, stearic acid salts, stearic acid derivatives, starch, hydrogenated vegetable oils, sodium benzoate, sodium acetate, leucine, PEG-4000 and magnesium lauryl sulfate, binders, buffering agents, antioxidants, chelating agents, surfactants, colorant, flavorant, sweetening agent, tablet antiadherents, diluent, excipient, opaquant, glidant, lubricant, polishing agent, pharmaceutically acceptable salts and combinations thereof.

In a another embodiment, a total daily dose of a formulation may be used as a dietary supplement is between about 1 mg to about 2000 mg of Annurca apple polyphenol extracts administered one or more times daily, e.g., two times, three times, four times, daily.

The dosage forms and compositions may comprise any of the forms and compositions known to the skilled artisan. In one embodiment, the sustained release formulation comprising Annurca apple polyphenol extracts is a tablet, capsule, gel or a liquid-soluble powder.

In one embodiment, the invention described herein includes the administration of a composition having one or more compounds extracted from a fruit or vegetable, specifically polyphenol extracted from Annurca apples as a dietary supplement for the prevention of cancer. In one embodiment, the mammal is a human.

The present invention may be used to treat carcinoma, glioma, astrocytoma, medulloblastoma, craniopharyngioma, ependymoma, pinealoma, fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma; leukemia, acute lymphocytic leukemia and acute myelocytic leukemia, myeloblastic leukemia, promyelocytic leukemia, myelomonocytic leukemia, monocytic leukemia, erythroleukemia, chronic leukemia, chronic myelocytic (granulocytic) leukemia, chronic lymphocytic leukemia, polycythemia vera, lymphoma, Hodgkin's disease, non-Hodgkin's disease, multiple myeloma, Waldenstrom's macroglobulinemia, and heavy chain disease.

In another embodiment, the polyphenolic composition of the present invention, along with a pharmaceutical carrier, may be used to treat neoplasia in a subject by affecting abnormal cell proliferation, modifying cell apoptosis, inducing cell cycle arrest, decreasing cell proliferation, modulation of epigenetic changes or combinations thereof.

The present invention provides a method of modulating cell proliferation by contacting cells with a pharmaceutical effective amount of the polyphenolic composition. Cell proliferation may be modulated in many ways, some examples include, but not limited to modifying apoptosis, inducting cell cycle arrest, decreasing cell proliferation, modulating epigenetic changes or a combinations thereof.

The present invention provides a method of treating colorectal cancer (CRC); however, other cancers and cells that can be treated by the polyphenolic composition include, but not limited to carcinoma cells, colorectal cancer cells, rectal carcinoma cells, hairy cell leukemia cells, osophogeal carcinoma cells, sarcoma cells, seminoma cells, angiosarcoma cells, carcinoma cells, chordoma cells, fibrosarcoma cells, myxosarcoma cells, liposarcoma cells, chondrosarcoma cells, osteogenic sarcoma cells, endotheliosarcoma cells, lymphangiosarcoma cells, lymphangioendotheliosarcoma cells, synovioma cells, mesothelioma cells, leiomyosarcoma cells, rhabdomyosarcoma cells, pancreatic cancer cells, breast cancer cells, ovarian cancer cells, prostate cancer cells, squamous cell carcinoma cells, basal cell carcinoma cells, adenocarcinoma cells, sweat gland carcinoma cells, sebaceous gland carcinoma cells, papillary carcinoma cells, papillary adenocarcinomas cells, cystadenocarcinoma cells, medullary carcinoma cells, bronchogenic carcinoma cells, renal cell carcinoma cells, hepatoma cells, bile duct carcinoma cells, choriocarcinoma cells, embryonal carcinoma cells, cervical cancer cells, testicular tumor cells, lung carcinoma cells, small cell lung carcinoma cells, bladder carcinoma cells, epithelial hemangioblastoma cells, acoustic neuroma cells, oligodendroglioma cells, meningioma cells, melanoma cells, neuroblastoma cells, retinoblastoma cells, acute lymphocytic leukemia cells, acute myelocytic leukemia cells, promyelocytic leukemia cells, myelomonocytic leukemia cells, monocytic leukemia cells, erythroleukemia leukemia cells, chronic myelocytic leukemia cells, chronic lymphocytic leukemia cells, polycythemia vera cells, lymphoma cells, Hodgkin's disease cells, non-Hodgkin's disease cells, multiple myeloma cells, Waldenstrom's macroglobulinemia cells, Ewing's tumor cells, Wilms' tumor cells or combinations thereof.

Yet in another embodiment, the polyphenolic composition is taken as a dietary supplement for the treatment or prevention of neoplasia and/or cancer in a subject. The present invention also provides a method of effecting the DNA methylation of colorectal cancer cells where the polyphenolic composition affects one or more methylation sites. In certain embodiments, the polyphenolic compound of the present invention, along with a pharmaceutical carrier, may be used in affecting neoplasia cells by inducing cell apoptosis, inducing tumor suppressor genes, and methylating cellular DNA.

In addition, the present invention may include administering in combination with other therapeutic agents, such as anti-cancer drugs, e.g., but are not limited to adriamycin and adriamycin conjugates, mechlorethamine, cyclophosphamide, ifosfamide, melphalan, chlorambucil, hexamethylmelamine, thiotepa, busulfan, carmustine, lomustine, semustine, streptozocin, dacarbazine, methotrexate, fluorouacil, floxuridie, cytarabine, mercaptopurine, thioguanine, pentostatin, vinblastine, vincristine, etoposide, teniposide, actinomycin D, daunorubicin, doxorubicin, bleomycin, plicamycin, mitomycin, L-asparaginase, interferon-alpha, cisplatin, carboplatin, mitoxantrone, hydroxyurea, procarbazine, mitotane, aminoglutethimide, prednisone, hydroxyprogesterone caproate, medroxyprogesterone acetate, megestrol acetate, diethylstilbestrol, ethinyl estradiol, tamoxifen, testosterone propionate, fluoxymesterone, flutamide, leuprolide, acetogenins, bullatacin, quassanoids, simalikalactone D, glaucarubolone, and pharmaceutically acceptable derivatives thereof.

The present invention may be used as a dietary or nutritional supplement for the prevention of cancer. In this embodiment, the total daily dose ranges of the active catechins for the conditions described herein are generally from about 1 mg to about 1000 mg administered in divided doses administered parenterally or orally. A preferred total daily dose is from about 50 mg to about 400 mg of the active extracts.

A kit for carrying out the therapeutic regimens of the invention comprise in one or more containers having therapeutically or prophylactically effective amounts of the Annurca apple polyphenol extract complexes in pharmaceutically acceptable form. The Annurca apple polyphenol extract complex in a vial of a kit of the invention may be in the form of a pharmaceutically acceptable solution, e.g., in combination with sterile saline, dextrose solution, or buffered solution, or other pharmaceutically acceptable sterile fluid. Alternatively, the complex may be lyophilized or desiccated; in this instance, the kit optionally further includes in a container a pharmaceutically acceptable solution (e.g., saline, dextrose solution, etc.), preferably sterile, to reconstitute the complex to form a solution for injection purposes. In another embodiment, a kit of the invention further comprises a needle or syringe, preferably packaged in sterile form, for injecting the complex, and/or a packaged alcohol pad. Instructions are optionally included for administration of Annurca apple polyphenol extract complex by a clinician or by the patient.

The present invention includes compositions and methods for modulating cell proliferation using a pharmaceutical effective amount of a polyphenolic composition having one or more polyphenolic compounds extracted from one or more plant tissues, specifically from one or more tissues of an Annurca apple. In some embodiments, the polyphenolic compounds include between 1 mM and 20 mM of each of catechins, chlorogenic acid and epicatechin.

The one or more polyphenolic compounds affects cell proliferation by a variety of mechanisms including modifying apoptosis, induction of cell cycle arrest, decrease in cell proliferation, modulation of epigenetic changes or a combination thereof.

The one or more polyphenolic compounds affects cell selected from the group of carcinoma cells, colorectal cancer cells, rectal carcinoma cells, hairy cell leukemia cells, osophogeal carcinoma cells, sarcoma cells, seminoma cells, angiosarcoma cells, carcinoma cells, chordoma cells, fibrosarcoma cells, myxosarcoma cells, liposarcoma cells, chondrosarcoma cells, osteogenic sarcoma cells, endotheliosarcoma cells, lymphangiosarcoma cells, lymphangioendotheliosarcoma cells, synovioma cells, mesothelioma cells, leiomyosarcoma cells, rhabdomyosarcoma cells, pancreatic cancer cells, breast cancer cells, ovarian cancer cells, prostate cancer cells, squamous cell carcinoma cells, basal cell carcinoma cells, adenocarcinoma cells, sweat gland carcinoma cells, sebaceous gland carcinoma cells, papillary carcinoma cells, papillary adenocarcinomas cells, cystadenocarcinoma cells, medullary carcinoma cells, bronchogenic carcinoma cells, renal cell carcinoma cells, hepatoma cells, bile duct carcinoma cells, choriocarcinoma cells, embryonal carcinoma cells, cervical cancer cells, testicular tumor cells, lung carcinoma cells, small cell lung carcinoma cells, bladder carcinoma cells, epithelial hemangioblastoma cells, acoustic neuroma cells, oligodendroglioma cells, meningioma cells, melanoma cells, neuroblastoma cells, retinoblastoma cells, acute lymphocytic leukemia cells, acute myelocytic leukemia cells, promyelocytic leukemia cells, myelomonocytic leukemia cells, monocytic leukemia cells, erythroleukemia leukemia cells, chronic myelocytic leukemia cells, chronic lymphocytic leukemia cells, polycythemia vera cells, lymphoma cells, Hodgkin's disease cells, non-Hodgkin's disease cells, multiple myeloma cells, Waldenstrom's macroglobulinemia cells, Ewing's tumor cells, Wilms' tumor cells and combinations thereof.

In some embodiments, the present invention may be used in conjunction with a therapeutically effective amount of one or more anticancer treatments (e.g., radiation therapy, chemotherapy, surgery, immunotherapy, photodynamic therapy, and a combination thereof) or antibiotic agent (e.g., doxorubicin, daunorubicin, epirubicin, idarubicin and anthracenedione, mitomycin C, bleomycin, dactinomycin, and plicatomycin).

The development of colorectal cancer has been described as a multistep model, where the accumulation of genetic and epigenetic events mediate the adenoma-carcinoma sequence⁹. The accumulation of mutations is driven through distinct pathways by different types of genomic instability, and the best characterized of these are called chromosomal instability (CIN) and microsatellite instability (MSI)¹⁰. These mechanistic pathways inactivate tumor suppressor genes by allelic loss and mutation, respectively. In addition, an epigenetic pathway provides the inactivation of tumor suppressor genes by promoter methylation and the silencing of gene transcription¹¹. This pathway has been called the CpG island methylator phenotype (CIMP). Although there is some degree of overlap among these pathways, evidence of CpG island methylator phenotype can be found in premalignant gastrointestinal epithelium 12, and is present in as many as 50% of all colorectal cancers^{13,14}. CpG island methylator phenotype is responsible for hypermethylation of the promoter of hMLH1 and subsequent MSI in approximately 12% of sporadic colorectal cancers¹⁵.

The CpG island methylator phenotype is a pathway wherein tumor suppressor genes are inactivated by promoter methylation and the silencing of gene transcription. The CpG island methylator phenotype, is a feature of up to 50% of colorectal cancers, and is characterized by DNA hypermethylation in the promoters of tumor suppressor genes with subsequent silencing of transcription. Polyphenols extracted from green tea and soybeans have been demonstrated to reverse hypermethylation in esophageal cancer models. For example, annurca apples, typical of southern Italy, are extremely rich in catechin, epicatechin, chlorogenic acid, and other polyphenols.

Colorectal cancer is the fourth commonest cancer and the third most common cause of cancer death in western countries¹. the present inventors recognized that the Mediterranean area is characterized by a lower incidence of cancers, including colorectal cancer². Although the overall incidence of colorectal cancer in Italy is comparable to that in continental European countries and the United States, a significant geographical north-south gradient exists³. Marked differences in the nutritional behavior occur across the country, in which the southern diet more fully reflects the Mediterranean model. This is compatible with the long-held belief that variations in nutrition are responsible for preventing cancer in certain geographical regions. The details of these relationships, and the mechanisms involved, however, have been difficult to identify.

For example, the Mediterranean diet, which is rich in fruits, vegetable, olive oil and red wine, is associated with a lower incidence of cancer^{4,5}. Although the cultural differences in the nutritional behavior are mostly identified in the macronutrient profiles, recent attention has been directed to specific “bioactive compounds” which are contained in plants products and lipid-rich fruits⁶. For example, phenolic compounds have a broad spectrum of properties including antineoplastic, antioxidant and anti-inflammatory⁷. Polyphenols are richly represented in many of the plants foods that form the basis of the Mediterranean diet.

Graziani and colleagues demonstrated that polyphenols extracted from Annurca apples can prevent exogenous damage in vitro to human gastric epithelial cells, and in vivo to rat gastric mucosa⁸. The Annurca apple is a variety with a “Protected Geographical Indication” of the Campania region in southern Italy; these apples are extremely rich in catechin, epicatechin and chlorogenic acid.

The development of colorectal cancer has been described as a multistep model, where the accumulation of genetic and epigenetic events mediate the adenoma-carcinoma sequence⁹. The accumulation of mutations is driven through distinct pathways by different types of genomic instability, and the best characterized of these are called chromosomal instability (CIN) and microsatellite instability (MSI)¹⁰. These mechanistic pathways inactivate tumor suppressor genes by allelic loss and mutation, respectively. In addition, an epigenetic pathway has been proposed wherein tumor suppressor genes are inactivated by promoter methylation and the silencing of gene transcription¹¹. This pathway has been called the CpG island methylator phenotype. Although there is some degree of overlap among these pathways, evidence of CpG island methylator phenotype can be found in premalignant gastrointestinal epithelium 12, and is present in as many as 50% of all colorectal cancers^{13,14} CpG island methylator phenotype is responsible for hypermethylation of the promoter of hMLH1 and subsequent MSI in approximately 12% of sporadic colorectal cancers¹⁵.

The regulation of DNA methylation involves DNA methyl transferases (DNMTs), which catalyze the transfer of methyl groups to the carbon-5 position of cytosines in CpG islands. DNMT-1 is largely responsible for maintaining methylation, and it also contributes to de novo DNA promoter methylation in cancer¹⁶. Some models suggest that DNMT-3b cooperates with DNMT-1 to maintain DNA methylation status¹⁷. Since epigenetic changes are highly relevant to colon carcinogenesis, and since this process is potentially reversible, it represents a target for the novel strategies to prevent or treat cancer. Moreover, the DNA methyltransferases inhibitors, 5-aza-cytidine and its metabolite 5-aza-2′deoxycytidine (5-aza-2dC) have been approved by the Food and Drug Administration for the treatment of myelodysplastic syndromes^{18,19}. However, serious side effects, including myelotoxicity, limit the use of these drugs in other clinical settings²⁰.

Interestingly, demethylating activities have been reported for certain tea catechins and soybean isoflavones in breast and esophageal squamous cell lines^{21,22}. The apparent safety and the easy access through the diet make these natural compounds attractive as chemopreventive and chemotherapeutic agents. Moreover, there is evidence that mixtures of bioactive compounds naturally present in foods may act synergistically, and might be more active than the solitary compounds isolated from food²³.

The present inventors recognized that Annurca apple polyphenols (APEs) provide anticancer properties in in-vitro models of colorectal cancer, e.g., DNA methylation (e.g., natural extracts and the synthetic DNMT inhibitor 5-aza-2dC) provides the ability to induce demethylation of DNA, reactivation of tumor suppressor genes, and the ability to modulate the expression of DNMTs.

Cell culture and treatments. Human CRC cell lines RKO, SW48 and SW480 were purchased from the American Type Culture Collection (ATCC). The cells were cultured in Iscove's Modified Dulbecco's Media (IMDM) supplemented with 10% fetal bovine serum (Life Technologies, Inc, Grand Island, N.Y.), 100 U/ml penicillin G, and 100 μg/mL streptomycin (GIBCO, Invitrogen Corporation, Carlsbad, Calif.). The cultures were maintained at 37° C. in 5% CO₂. Annurca apple polyphenols were extracted from the frozen flesh of Annurca apples as previously described⁸. 5-aza-2dC was purchased from Sigma Aldrich (St. Louis, Mo.). Cells were treated with Annurca apple polyphenol extract or 5-aza-2dC; 5-aza-2dC treatment a final concentration of 5 μM for 96 hours, changing the conditioned media each day. Annurca apple polyphenol extract treatment was performed based on the IC₅₀ results by changing the conditioned media every 48 hours.

Cell viability (MTT assay). Cells were seeded at a density of 3000 cell/well in 96-well plates. The next day, cells were treated with concentrations ranging from between about 0 to about 10 μM of Annurca apple polyphenols dissolved in methanol. Appropriate amounts of methanol were used in the control wells. After 24 and 96 hours of treatment, the cells were incubated with a solution of MTT [3-(4,5-dimethylthiazol-2-yl)-2,5-diphenyltetrazolium bromide] (Sigma, St Louis, Mo.) at a concentration of 0.5 μg/μL for 3 h at 37° C. The cells were lysed in 100 μL of solubilizing solution (10% SDS, 0.01 N HCl). Colored formazan converted from MTT by viable cells was measured at 570 nm using a microplate reader.

Determination of the induction of apoptosis. Late apoptotic events were analyzed by terminal transferase dUTP nick end labeling (TUNEL) assay using the In-Situ Cell Death Detection Kit (Roche, Branchburg, N.J.). Briefly, cells were grown on glass cover slips in 24 well plates at a concentration of about 3×10³ cells/well, followed the next day by 96 hours treatments with Annurca apple polyphenols at a final concentration of 2 μM, changing the conditioned media every 48 hours. Equal amounts of methanol were used in the control wells. TUNEL assays were performed according to the manufacturer's protocol. Pretreatment with DNase 1 (3000 U/mL in 50 mM Tris-HCl, 1 mg/ml BSA) was used for the positive controls. Apoptotic cells were visualized under an AxioSkop2 multichannel epifluorescence microscope and processed by AxioVision software (Carl Zeiss Inc., Thornwood, N.Y.).

Early apoptotic events were analyzed using the Annexin V-FITC detection kit (Pharmingen, San Diego, Calif.) according to the protocol suggested by the manufacturer. Briefly, the cells were plated at a density of 1×10⁶ in 100-mm dishes and treated with APEs for two days. After treatment, the cells were harvested and washed twice in PBS. 10⁶ cells were suspended in 100 μL 1× binding buffer (10 mM Hepes/NaOH pH 7.4; 140 mM NaCl; 2.5 mM CaCl₂) and stained with 2.5 μL of annexinV-FITC and 5 μL of 7-amino-actinomycin-D (7-AAD). The cell suspensions were gently vortexed and incubated for 15 minutes at 4° C. in the dark. After adding 400 μL of binding buffer, the cells were analyzed by FACScalibur Flow Cytometer (Becton Dickinson, Franklin Lakes, N.J. USA). Unstained cells and cells stained with Annexin V-FITC or 7-AAD were used for florescence compensation.

Cell cycle analysis. The effects of Annurca apple polyphenols on cell cycle profiles were evaluated by flow cytometry. Cell cycle distribution was based on an evaluation of the amount of the DNA stained with propidium iodide (PI). Cells were plated at a density of 5×10⁵ cells/plate in 100 mm dishes, synchronized by serum deprivation for 48 hours and finally treated with 2 μM of APEs, with the conditioned media changed every other day, for a total duration of 96 hours. The cells were harvested, resuspended at a density of 5×10⁶ cells/mL in cold PBS and fixed with 80% ethanol overnight at −20° C. The next day, the cells were washed, resuspended in 300 μL of PBS, incubated with 160 μg/mL of boiled and renatured ribonuclease A (RNase A) for 15 minutes at 37° C. and stained with 80 μg/mL of PI for 30 minutes. DNA content was evaluated by a FACScalibur Flow Cytometer (Becton Dickinson, Franklin Lakes, N.J.). Cell Cycle distribution was determined using the ModFit DNA Analysis Software (Verity Software House, Topsham, Me.).

Western blotting analysis. Protein expression was assessed by western blotting. Protein extraction was performed using radioimmunoprecipitation (RIPA) Buffer (Santa Cruz Biotechnology, Santa Cruz, Calif.) combined with 10 μl/mL of phenylmethylsulfonyl fluoride (PMSF) solution, 10 μL/mL sodium orthovanadate solution and 10 μL/mL protease inhibitor cocktail. The appropriate amount of lysis buffer was added to each sample and the pellets were sheared with a syringe. The cell lysates were incubated for 1 hour on ice and centrifuged for 10 minutes to obtain clear supernatants. The protein concentration was measured by BCA using the BCA protein assay kit (Pierce, Rockford, Ill.) as indicated by the manufacturer. Forty μg of proteins were separated on 10% sodium dodecyl sulfate-polyacrylamide gel electrophoresis (SDS-PAGE) gels. The separated proteins were transferred onto PVDF membrane (Amersham Pharmacia Biotech). Transferred proteins were stained with Ponceau Red to confirm successful transfer, followed by blocking with 5% non-fat milk powder in TBS-T (50 mM Tris, pH 7.6, 150 mM NaCl, 0.1% Tween-20). Membranes were then probed with the specific primary antibody followed by incubation with a peroxidase-conjugated anti-mouse secondary antibody (0.08 μg/mL; Santa Cruz) for 30 minutes. The protein bands were visualized using the ECL Plus Chemiluminescence system, and the membranes were scanned with a STORM 840 Phosphorimager (Amersham Biociences Inc., Arlington Heights, Ill.). Quantification of the bands was performed using IMAGEQUANT 5.2 spot densitometric software (Molecular Dynamics, Sunnyvale, Calif.). The expression levels of the proteins were corrected by normalization to the expression of the housekeeping protein β-actin. Cell cycle protein primary antibodies including anti-cyclin D1 (clone A-12), anti-cyclin E (clone HE 12), anti-cyclin B1 (clone D11), anti-p53 (clone DO-1), and anti-p21 (clone F5) were obtained from Santa Cruz Biotechnology (Santa Cruz Biotechnology, Santa Cruz, Calif.) and incubated for 3 hours at room temperature. Anti-hMLH1 and anti-DNMT-1 antibodies were purchased from BD Biosciences Pharmingen (San Diego, Calif.) while the anti-DNMT-3b antibody was purchased from Imgenex (San Diego, Calif.). These antibodies were incubated overnight at 4° C. All antibodies were used at a working concentration of 2 μg/mL.

Bisulfite modification, methylation specific PCR (MSP) and combined bisulfite restriction assay (COBRA). After treatment with APEs or 5-aza-2dC, DNA extraction was performed using the QIAamp DNA Mini kit (Qiagen, Valencia, Calif.) and 500 ng of DNA were subjected to bisulfite modification with the Epitect Bisulfite Kit (Qiagen, Valencia, Calif.) as recommended by the manufacturer's protocol. Modified DNA was used as a template. The status of hMLH1 promoter methylation was assessed by MSP-PCR, while p14^{ARF} and p16^{INK4a} promoter methylation were assessed by COBRA, as previously described^{24,25,26}.

Real-time and conventional reverse transcriptase (RT) PCR. After treatment with APEs or 5-aza-2dC, total RNA was extracted using Trizol reagents (Invitrogen, Carlsbad, Calif.) according to the manufacturer's instructions. TaqMan One step RT-PCR Master Mix (Roche, Branchburg, N.J.) and a TaqMan Gene Expression Assay for hMLH1 (Cat # Hs 00179866; Applied Biosystems, Foster City, Calif.) were used. One microgram of total RNA from each sample was used as a template. GAPDH was used as an endogenous control. The ABI Prism 7000 Sequence Detection System (Applied Biosystems, Foster City, Calif.) was used for real-time PCR analysis. Thermal cycling conditions were designed as follows: RNA retro-transcription at 48° C. for 30 minutes followed by initial denaturation at 95° C. for 10 minutes, 40 cycles of 95° C. for 15 seconds, and 60° C. for 1 minutes.

Conventional RT-PCR was also performed to assess the mRNA expression of p14^{ARF 27}, p16^{INK4a 28}, hMLH1²⁹, DNMT-1³⁰ and DNMT-3b³¹. cDNA was generated with random hexamers using 2 μg of RNA. The PCR was performed as previously reported³². β-actin was used as an endogenous control. The statistical significance of differences between the experimental points or between groups was analyzed using Student's t-test; and differences were considered significant when P<0.05.

Annurca apple polyphenols inhibit cell viability in RKO and SW480 cell lines. RKO and SW48 are CpG island methylator phenotype models, and both have undergone silencing of the DNA mismatch repair gene hMLH1 and subsequently developed MSI. The tumor suppressor genes p14^{ARF} and p16^{INK4a} are also methylated in both lines¹⁵. SW480 cells, on the other hand, are a model of CIN, do not have CpG island methylator phenotype features, and the DNA mismatch repair system is fully functional in these cells.

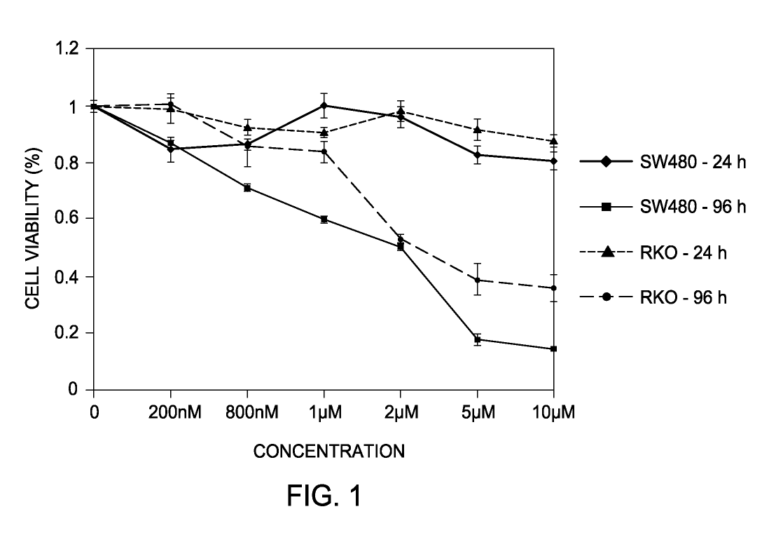

FIG. 1 is a graph of the percentage of cell viability in RKO and SW480 cell lines after Annurca apple polyphenol treatment for 24 hours as seen in the upper profile and 96 hours as seen in the lower profile using the MTT assay. Annurca apple polyphenol concentrations are given as catechin equivalent. The values of cell viability are expressed as a ratio between the absorbance of treated cells and untreated controls. Each point is from data in triplicate with p<0.05 in RKO and p<0.05 in SW480.

The effect of Annurca apple polyphenol treatment on cell viability was determined by MTT assay. The cells were treated with different concentrations of Annurca apple polyphenol extract (e.g., 0-10 μM catechin equivalent) for 24 and 96 hours as seen in FIG. 1. The treatment reduced the viability of RKO and SW480 cell lines in a dose- and time-dependent manner. No changes in the cell viability were detected after just 24 hours at any concentration, whereas significant decreases in cell viability were evident after 96 hours of treatment at concentrations of 2 μM for RKO and 200 nm for SW480 the cell lines with a p<0.05 as seen in FIG. 1. At 2 μM, cell viability was decreased by 49±0.03% for SW480 and by 47±0.027% for RKO.

Annurca apple polyphenols induce apoptosis in RKO cells (early and late stage) and SW480 cells (late stage). Apoptosis was evaluated either by changes in the membrane permeability using Annexin-V/7-AAD assay (early marker) and by DNA fragmentation using TUNEL assay (late marker). The annexin V/7-AAD assay was carried out after 24 hours of treatment.

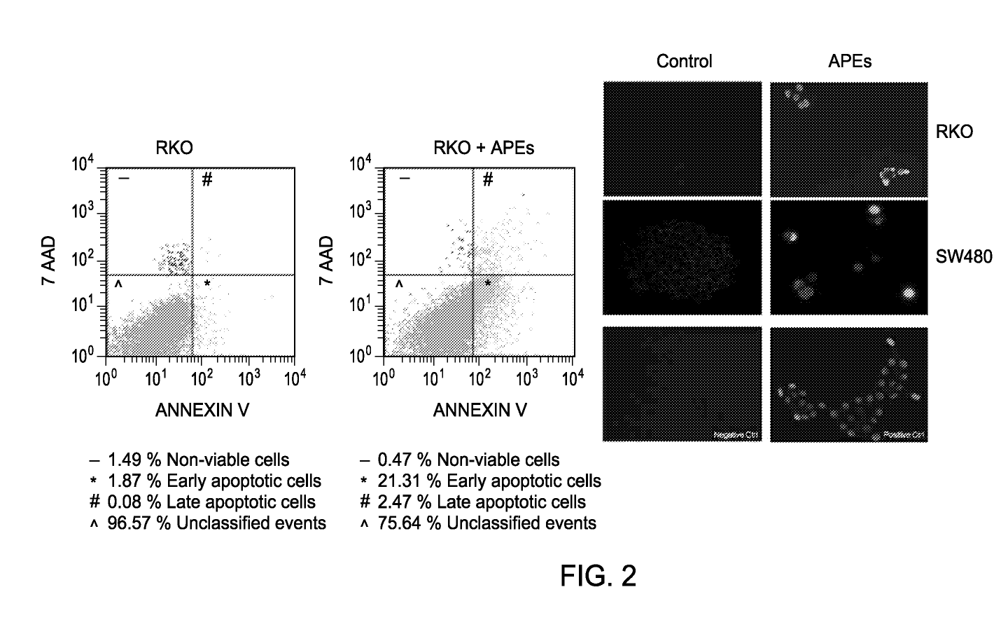

FIGS. 2A and 2B are plots of the apoptotic response to Annurca apple polyphenol treatment. FIG. 2A is a plot of the percentage of apoptotic response to Annurca apple polyphenol treatment evaluated by an annexinV/7AAD flow cytometry assay. Early apoptotic cells stain annexin V+ and 7AAD− while late apoptotic/necrotic cells stain annexin V+ and 7AAD+ with the viable cells being annexin V-7AAD-. A significant increase of early apoptotic cells was evident in RKO after 24 hours of Annurca apple polyphenol treatment (p<0.001). No changes in the early apoptotic cell population were detectable in SW480.

FIG. 2B is an image of a DNA fragmentation as a marker of late apoptosis evaluated by the TUNEL assay. RKO and SW480 cell lines were treated for 96 hours. Several TUNEL positive cells were revealed in the treated samples for both cell lines. No staining was found in the controls. Pretreatment with DNase I was used as a positive control and incubation without terminal deoxynucleotidyltransferase, the enzyme that catalyzes the incorporation of labeled nucleotides to the fragmentized DNA, was performed in the negative control.

As shown in FIG. 2A, Annurca apple polyphenols induced a significant increase in the early apoptotic cell population in RKO after treatment (e.g., 1.87% vs 21.31%, p<0.001), while changes were not found in SW480 (data not shown). Subsequently, the late phases of the apoptosis were evaluated by TUNEL assay after 4 days of treatment. As shown in FIG. 2B, apoptotic cells were visible after treatment of either SW480 or RKO, while no staining was detectable in the negative controls.

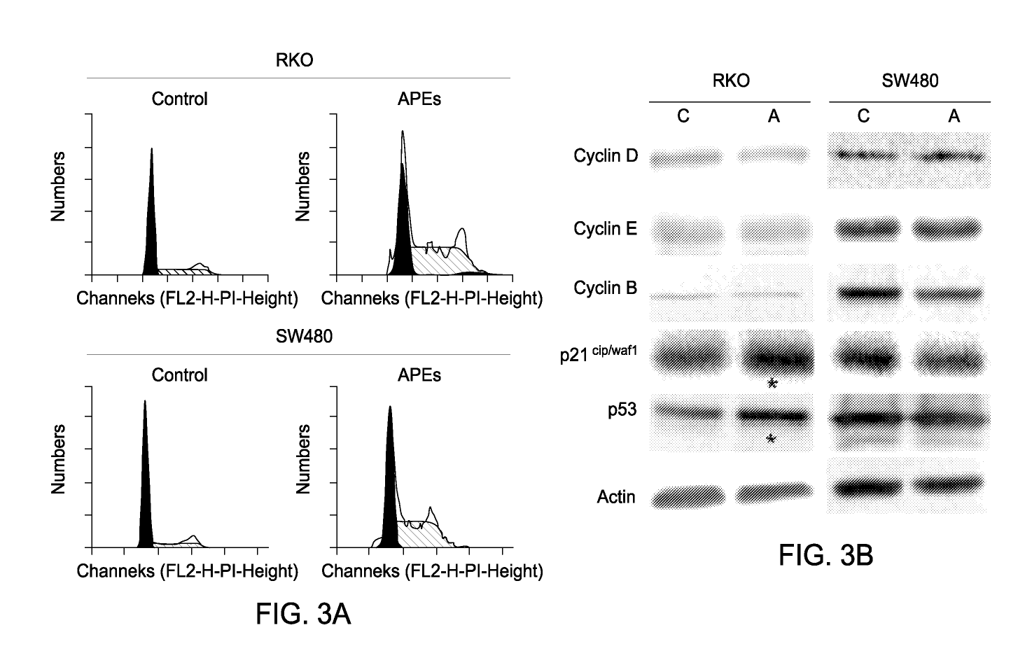

FIG. 3 illustrates the effects of Annurca apple polyphenol treatment in cell cycle dynamics. FIG. 3A is a plot of a flow cytometric analysis of the cell cycle distribution in Annurca apple polyphenol extract-treated samples and untreated controls in RKO and SW480 cells. A non-significant arrest in S phase was apparent in the FACS-scan profile. FIG. 3B is an image of a Western blot of cell cycle regulatory proteins before and after Annurca apple polyphenol extract treatment in RKO and SW480 cell lines. Significant changes were observed in the expression p21^{cip/waf} and p53 in RKO after Annurca apple polyphenol extract treatment, e.g., p<0.05. The effects of Annurca apple polyphenols on the cell cycle were evaluated by flow cytometry after staining the cells with propidium iodide. Cells were synchronized by serum deprivation and subsequently treated with Annurca apple polyphenols for 4 days. At flow-cytometry, the cell cycle profile showed a slight S phase-arrest in both RKO and SW480 (see, e.g., FIG. 3A). To confirm these results, the level of the expression of the principal cell cycle regulatory proteins was assessed by western blot (see e.g., FIG. 3B). No significant changes in the expression of cyclins D, E or B were detected after treatment. Significant increases in p53 and its downstream target p21^{cip/waf} were obtained in RKO cells (p<0.05).

Annurca apple polyphenols induce reversal of methylation and reactivation of hMLH1, p14^{ARF} and p16^{INK4a} Based on reported data of demethylating activity on esophageal squamous cell carcinoma after treatment with epigallocatechin-3 gallate extracted from green tea³³ and soy isoflavones²², the effects of Annurca apple polyphenol extracts on the DNA methylation status of colorectal cancer cells was investigated. Cells were treated with 2 μM Annurca apple polyphenol extract for 4 days.

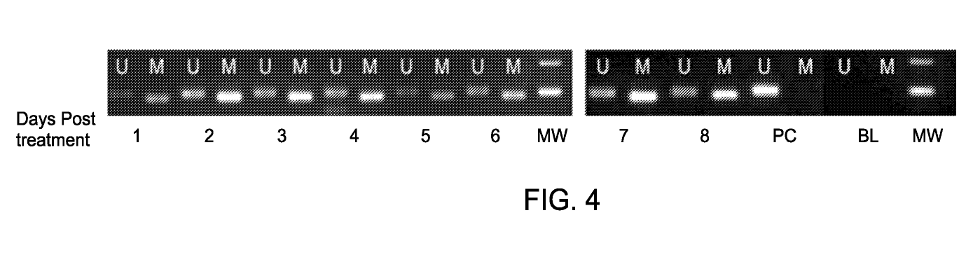

FIG. 4 evaluation of the methylation status of the hMLH1 promoter by methylation specific PCR (MSP) in RKO cells, which are normally fully methylated. A specific unmethylated band was amplified starting from the second day after treatment. The effects on methylation were evident at least until the eighth day after the end of treatment. Human placental DNA treated in vitro with SssI methylase was used as a positive control for MSP of methylated alleles (PC), where BL is the blank and MW is the size standard. Following treatment with Annurca apple polyphenols, MSP of the hMLH1 promoter revealed the appearance of the specific unmethylated band in RKO cells, from the second day after initiation of treatment until the eighth day as seen in FIG. 4.

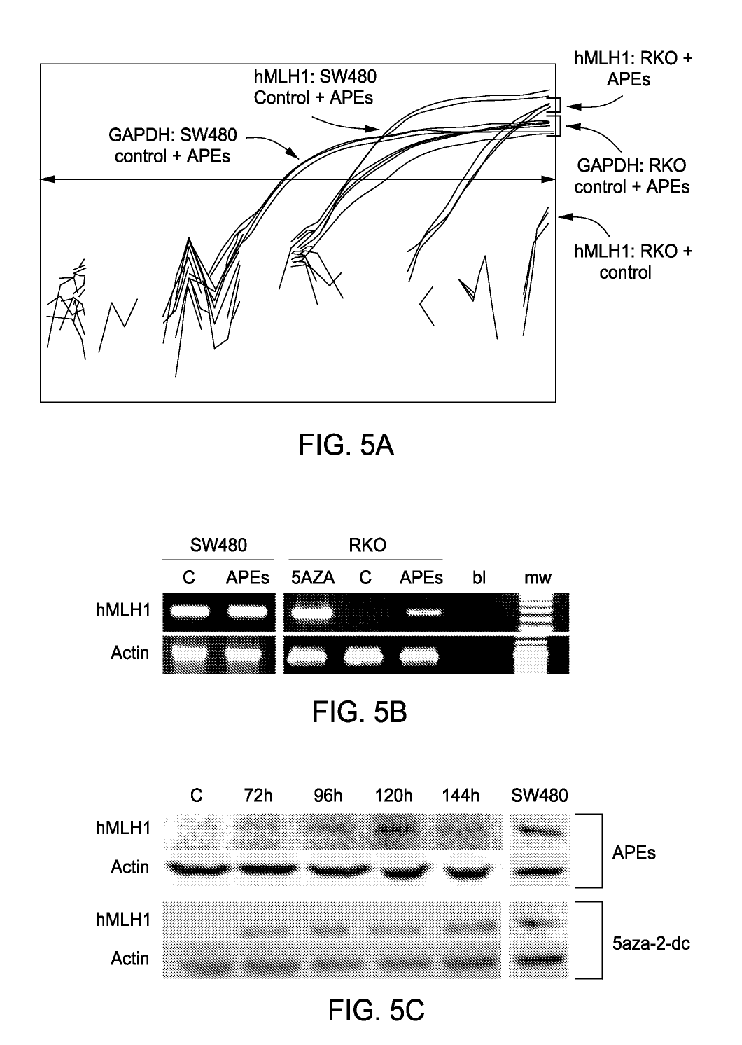

FIGS. 5A, 5B and 5C are images illustrating hMLH1 mRNA expression. FIG. 5A is a graph of the re-expression of hMLH1 transcripts by real time PCR in response to Annurca apple polyphenol extract treatment in RKO cells. FIG. 5B is an image of a conventional RT-PCR amplification of hMLH1 in RKO after treatment with either 5-AZA-2dc or Annurca apple polyphenols. SW480 was used as positive control for hMLH1 expression. FIG. 5C is an image a western blot of hMLH1 protein expression in RKO. Increased protein expression was present by the third day after the end of the treatment, peaking at the fifth day post treatment. Although lasting for a longer time, a similar pattern of re-expression was obtained after treatment with 5-aza-2dC.

The demethylating effect resulted in the re-expression of hMLH1 mRNA assessed either by conventional real time PCR or real time PCR as seen in FIGS. 5A and 5B. The methylated promoter band was still visible during the appearance of the unmethylated band. These results confirm that total demethylation of the gene promoter is not necessary for re-expression of the transcripts or protein³⁴. In fact, western blot analysis performed for hMLH1 protein showed increasing expression of the protein from the third day after the termination of treatment, and peaking at the fifth day. The reversal of hMLH1 DNA hypermethylation and re-expression of the mRNA and protein were comparable in magnitude to results obtained after treating the cells with 5-aza-2dc as seen in FIG. 5C. No changes in the expression profiles were observed in SW480 after treatment.

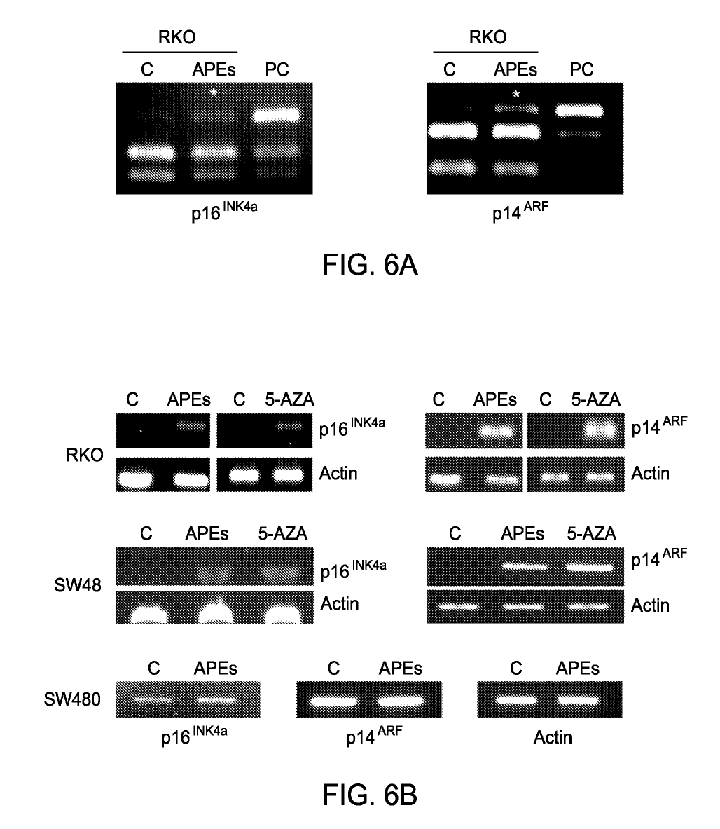

FIGS. 6A-6B are images of gels illustrating the re-expression of previously silenced tumor suppressor genes by demethylation. FIG. 6A is an image of the gel that evaluates p16^{INK4} a and p14^{ARF} methylation by COBRA in RKO. The appearance of the unmethylated band is demonstrated after Annurca apple polyphenol treatment and PC indicates human placental DNA with SssI methylase, as a positive control.

FIG. 6B is an image of a gel that examines the re-expression of p16^{INK4a} and p14^{ARF} mRNA was obtained in RKO cells after either Annurca apple polyphenols or 5-aza-2dC treatment; re-expression of p16^{INK4a} and p14^{ARF} mRNA in SW48 after either APE or 5-aza-2dC treatment, indicating that the effects of Annurca apple polyphenols are not cell-specific and SW480 was employed as a positive control for both genes.

The DNA methylation status of the promoters of p14^{ARF} and p16^{INK4a} were also investigated, and both were methylated in RKO^{35,36} by COBRA. Treatment with Annurca apple polyphenols resulted in the appearance of an unmethylated band for both promoters as seen in FIG. 6A, followed by an increase in RNA transcripts. This was comparable with the results obtained after treating the cells with 5-aza-2-dc see FIG. 6B. The transcript levels of p14^{ARF} and p16^{INK4a} were also evaluated in SW48 cells, which are CpG island methylator phenotype-positive. The appearance of RNA transcripts for both genes occurred in both the Annurca apple polyphenols and 5-aza-2dC treated samples, as seen in FIG. 6B.

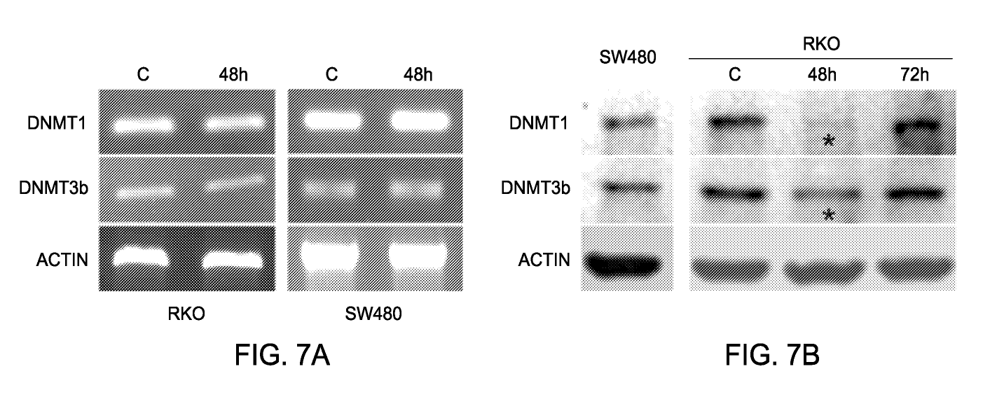

FIGS. 7A and 7B are images of gels illustrating the expression of DNMT genes after Annurca apple polyphenol treatment in RKO cells. Inhibition of DNA methyltransferase-1 and -3b by Annurca apple polyphenols is illustrated in FIG. 7 where expression of DNMT genes after Annurca apple polyphenol treatment in RKO cells. FIG. 7A is an image of the DNMT-1 and DNMT-3b mRNA expression evaluated by RT-PCR. No change in mRNA was observed after treatment in either RKO or SW480 cells. FIG. 7B is an image of DNMT-1 and DNMT-3b protein expression were evaluated by western blot. A significant decrease in DNMT-1 (p<0.001) and DNMT-3b (p<0.005) was evident 48 hours after the end of the treatment with Annurca apple polyphenol extract in RKO cells.

Finally, to clarify the mechanism by which Annurca apple polyphenols induce DNA demethylation changes in DNMT levels induced by Annurca apple polyphenols were examined. For this purpose, expressions DNMT-1 and DNMT-3b mRNA and protein were assessed. No differences in the transcript levels were detected between treated and control cells as seen in FIG. 7A, while a significant reduction in protein expression was found 48 hours after the end of the treatment for both DNMT-1 (p<0.001) and DNMT-3b (p<0.005) (as seen in FIG. 7B). The time course of the inhibition of DNMTs was consistent with the timing of promoter demethylation as well as re-expression of both RNA and protein of the tumor suppressor genes that tested. Taken together, these results suggest that Annurca apple polyphenols induce de-methylation through a post-translational inhibition of both DNMT-1 and DNMT-3b.

The present invention provides polyphenols extracted from Annurca apples, containing chlorogenic acid, catechin and epicatechin as major components, are active in regulating apoptosis and cell viability, without exerting significant effects on cell cycle dynamics in two different models of colorectal cancer: RKO (MSI and CIMP+) and SW480 (CIN and CIMP−). More importantly, our results show that Annurca apple polyphenols lead to the reactivation of silenced tumor suppression genes by inhibition of DNMT-1 and DNMT-3b protein expression in two CpG island methylator phenotype models of colorectal cancer (RKO and SW48 cells). Interestingly, our results with Annurca apple polyphenols were similar in magnitude to what we achieved with the known therapeutic compound, 5-aza-2dC, a potent but toxic synthetic DNMT inhibitor. Moreover, these findings are compatible to what has been observed with other nutriceuticals such as tea catechins and soy bioflavonoids, each of which can modulate DNMT-1 protein levels^{37,38}. The present invention provides a biological response with well-tolerated dietary substances including at least one variety of apples.

Polyphenols comprise a large family of compounds that encompass more than 8000 identified phenolic structures. The anticancer properties of these compounds have been widely investigated in a variety of tumors and models. The anticancer effects are compound-dependent, ranging from effects on apoptosis, induction of cell cycle arrest, decrease in cell proliferation and modulation of epigenetic changes²³. Polyphenols are present in virtually all plant-derived foods, and they represent key components of the Mediterranean diet, which is rich in vegetables, fruits, nuts, seeds, olive oil, grains, wine, and honey^{6,39-41}. The Mediterranean diet is universally associated with lower incidences of cancer, including colorectal cancer^{2,40}. This study focused on the anticancer effects of the polyphenols present in the Annurca apple, because it is found in a specific area of southern Italy with less colorectal cancer, and extracts from this apple have already been demonstrated to protect against exogenous gastric damage⁸.

Since the spectrum of anticancer properties of polyphenols is compound-specific, we first tested the anticancer activities of Annurca apple polyphenols were mediated by effects on cell viability, apoptosis and/or the cell cycle. The Annurca apple polyphenols inhibit cell viability in a time- and concentration-dependent manner. However, no important changes in cell cycle dynamics were found in either model. We observed the induction of apoptosis after Annurca apple polyphenols treatment in both models, including the induction of early and late apoptosis in RKO, and late apoptosis in SW480, e.g., the activity of Annurca apple polyphenols on the induction of p53-induced apoptosis. RKO has a fully functional p53 system⁴² and the western blot analysis performed after treatment with Annurca apple polyphenols showed a significant increase in both p53 and the p53 downstream target, p21^{waf1/cip1}.

Annurca apple polyphenols have an effect on the DNA methylation status of colorectal cancer cells. Tea EGCG³³ and soy isoflavones²² have been demonstrated to reverse the methylation of p16^{INK4a}, RARβ and other genes, and it is thought that this is mediated through the inhibition of DNMT-1. The natural combination of polyphenols present in Annurca extracts were used. The main phenolic compounds of Annurca apple polyphenol extracts are catechins (11.9 mg/100 g of apple flesh), chlorogenic acid (9.1 mg/100 g) and epicatechin (6.3 mg/100 g). These extracts have been demonstrated to prevent exogenous gastric damage in vitro and in vivo, and protective antioxidant effects have been obtained at concentrations of 2.5 mM and 10 mM respectively⁴³. Significantly lower concentrations were sufficient to obtain a potent demethylating effect with subsequent re-expression of previously silenced tumor suppressor genes. This observation indicates that the demethylating activity could be the key protective mechanism of these compounds. In silico molecular modeling studies have demonstrated that structural analogues of EGCG may interact with the catalytic domain of DNMT-1³³. An additional demethylating mechanism is mediated by the inhibitory feedback of S-adenosyl-homocysteine (SAH) on DNMTs. Several catechol-containing dietary polyphenols are excellent substrates for catechol-O-methyltransferase (COMT) mediated O-methylation that occurs in parallel with DNMT-mediated methylation. COMT methylation is responsible for reducing the cellular pool of S-adenosyl-methionine (SAM), substrates for both enzymes, and increasing SAH levels with negative feedback on the DNMT pathway³⁷. Using synthetic compounds, it has been demonstrated that catechin and epicatechin are DNMT inhibitors. Although EGCG seems to be more potent as a direct inhibitor, catechin and epicatechin are better substrates for COMT methylation with stronger negative feedback on the DNMTs, and better intracellular bioavailability^{37,44}. Nevertheless, our data on the expression of DNMTs show suppression at the protein level, whereas no differences were evident on the transcripts of either DNMT-1 or DNMT-3b, suggesting that the post-translational inhibition may represent the main mechanism of Annurca apple polyphenol extract treatment in this pathway. Strong DNMT protein inhibition occurred by the second day after Annurca apple polyphenol extract treatment, and the time course of DNMT inhibition coincided with the timing of promoter demethylation, mRNA appearance and protein re-expression of the previously silenced genes, and these effects were robust. The unmethylated DNA band and re-expression of the hMLH1 mRNA were evident until the 8^{th} day after the end of the treatment, and protein expression was evident for at least 6 days after the cessation of treatment.

Additionally, the demethylating effects of Annurca apple polyphenols were compared with the clinically used DNMT inhibitor 5-aza-2dC. Comparing the biological effects of the Annurca apple polyphenols versus 5-aza-2dC treatment, indicated that Annurca apple polyphenols can inhibit DNMT expression and reactivate silenced genes (hMLH1, p14^{ARF} and p16^{INK4a}) at very low concentration in two different CpG island methylator phenotype+colorectal cancer models (RKO and SW48). Demethylating activity of Annurca apple polyphenols compared with 5-aza-2′dC in vitro is consistent with previous reports on EGCG⁴⁵, excessive inhibition of DNMTs might be dangerous, as it has been associated with the induction of chromosomal instability in vitro, and sarcomas and T-cell lymphoma in vivo^{46,47}. There has never been a suggestion of toxicity due to excessive hypomethylation in individuals who regularly consume apples. Although issues of dose and tissue distribution make speculation difficult here, it would be hard to imagine that a commonly consumed variety of apples are toxic.

Promoter hypermethylation of tumor suppressor genes can be found in up to about 50 percent of colorectal cancers^{11,14}. Hypermethylation of tumor suppressor gene promoters is now considered among the earliest events occurring in colon carcinogenesis, and it can be found prior to the histological appearance of neoplasia 48. To date, no demethylating drugs are available for the treatment of colon cancer patients, or perhaps more importantly, for its chemoprevention. Currently, 5-aza-cytidine and its metabolite 5-aza-2-dC have been approved for the treatment of myelodysplasia,¹⁸ and several clinical trials are currently being conducted. Although promising results have been achieved on the hematological malignancies, the limited clinical responses^{49,51} and the spectrum of side effects have discouraged the use of these agents in other tumors^{19,49,52}. Nevertheless, the reactivation of hypermethylated tumor suppressor genes still remains an attractive strategy for cancer therapy.

In some geographical areas, Annurca apples have long been consumed as a dietary staple, and their long-term effects (in addition, to other protective nutrients of the Mediterranean diet) may be reflected in the lower incidences of cancer, including colorectal cancer. The present invention provides Annurca apple polyphenols adapted as a preventive and/or therapeutic armamentaria against colorectal cancer.

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, MB, BBC, AAABCCCC, CBBAAA, CABABB, and so forth. The skilled artisan will understand that typically there is no limit on the number of items or terms in any combination, unless otherwise apparent from the context.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

### REFERENCES

1. Jemal A, Murray T, Ward E, Samuels A, Tiwari R C, Ghafoor A, Feuer E J, Thun M J. Cancer statistics, 2005. CA Cancer J Clin 2005; 55:10-30.
2. Berrino F, Muti P. Mediterranean diet and cancer. Eur J Clin Nutr 1989; 43 Suppl 2:49-55:49-55.
3. Capocaccia R, De A R, Frova L, Gatta G, Sant M, Micheli A, Berrino F, Conti E, Gafa L, Roncucci L, Verdecchia A. Estimation and projections of colorectal cancer trends in Italy. Int J Epidemiol 1997; 26:924-932.
4. Berrino F, Muti P. Mediterranean diet and cancer. Eur J Clin Nutr 1989; 43 Suppl 2:49-55:49-55.
5. Gallus S, Bosetti C, La V C. Mediterranean diet and cancer risk. Eur J Cancer Prev 2004; 13:447-452.
6. Kris-Etherton P M, Hecker K D, Bonanome A, Coval S M, Binkoski A E, Hilpert K F, Griel A E, Etherton T D. Bioactive compounds in foods: their role in the prevention of cardiovascular disease and cancer. Am J Med 2002; 113 Suppl 9B:71S-88S:71S-88S.
7. Scalbert A, Williamson G. Dietary intake and bioavailability of polyphenols. J Nutr 2000; 130:2073 S-2085S.
8. Graziani G, D'Argenio G, Tuccillo C, Loguercio C, Ritieni A, Morisco F, Del Vecchio B C, Fogliano V, Romano M. Apple polyphenol extracts prevent damage to human gastric epithelial cells in vitro and to rat gastric mucosa in vivo. Gut 2005; 54:193-200.
9. Fearon E R, Vogelstein B. A genetic model for colorectal tumorigenesis. Cell 1990; 61:759-767.
10. Grady W M. Genomic instability and colon cancer. Cancer Metastasis Rev 2004; 23:11-27.
11. Herman J G, Baylin S B. Gene silencing in cancer in association with promoter hypermethylation. N Engl J Med 2003; 20; 349:2042-2054.
12. Rashid A, Shen L, Morris J S, Issa J P, Hamilton S R. CpG island methylation in colorectal adenomas. Am J Pathol 2001; 159:1129-1135.
13. Goel A, Arnold C N, Niedzwiecki D, Chang D K, Ricciardiello L, Carethers J M, Dowell J M, Wasserman L, Compton C, Mayer R J, Bertagnolli M M, Boland C R. Characterization of sporadic colon cancer by patterns of genomic instability. Cancer Res 2003; 63:1608-1614.
14. Goel A, Nagasaka T, Arnold C N, Inoue T, Hamilton C, Niedzwiecki D, Compton C, Mayer R J, Goldberg R, Bertagnolli M M, Boland C R. The CpG island methylator phenotype and chromosomal instability are inversely correlated in sporadic colorectal cancer. Gastroenterology 2007; 132:127-138.
15. Toyota M, Ahuja N, Ohe-Toyota M, Herman J G, Baylin S B, Issa J P. CpG island methylator phenotype in colorectal cancer. Proc Natl Acad Sci USA 1999; 20; 96:8681-8686.
16. Jair K W, Bachman K E, Suzuki H, Ting A H, Rhee I, Yen R W, Baylin S B, Schuebel K E. De novo CpG island methylation in human cancer cells. Cancer Res 2006; 66:682-692.
17. Rhee I, Bachman K E, Park B H, Jair K W, Yen R W, Schuebel K E, Cui H, Feinberg A P, Lengauer C, Kinzler K W, Baylin S B, Vogelstein B. DNMT1 and DNMT3b cooperate to silence genes in human cancer cells. Nature 2002; 416:552-556.
18. Mack G S. Epigenetic cancer therapy makes headway. J Natl Cancer Inst 2006; 98:1443-1444.
19. Issa J P. DNA methylation in the treatment of hematologic malignancies. Clin Adv Hematol Oncol 2005; 3:684-686.
20. Lyko F, Brown R. DNA methyltransferase inhibitors and the development of epigenetic cancer therapies. J Natl Cancer Inst 2005; 19; 97:1498-1506.
21. Fang M Z, Wang Y, Ai N, Hou Z, Sun Y, Lu H, Welsh W, Yang C S. Tea polyphenol (−)-epigallocatechin-3-gallate inhibits DNA methyltransferase and reactivates methylation-silenced genes in cancer cell lines. Cancer Res 2003; 63:7563-7570.
22. Fang M Z, Chen D, Sun Y, Jin Z, Christman J K, Yang C S. Reversal of hypermethylation and reactivation of p16INK4a, RARbeta, and MGMT genes by genistein and other isoflavones from soy. Clin Cancer Res 2005; 11:7033-7041.
23. Surh Y J. Cancer chemoprevention with dietary phytochemicals. Nat Rev Cancer 2003; 3:768-780.
24. Herman J G, Graff J R, Myohanen S, Nelkin B D, Baylin S B. Methylation-specific PCR: a novel PCR assay for methylation status of CpG islands. Proc Natl Acad Sci USA 1996; 93:9821-9826.
25. Shen L, Ahuja N, Shen Y, Habib N A, Toyota M, Rashid A, Issa J P. DNA methylation and environmental exposures in human hepatocellular carcinoma. J Natl Cancer Inst 2002; 94:755-761.
26. Herman J G, Umar A, Polyak K, Graff J R, Ahuja N, Issa J P, Markowitz S, Willson J K, Hamilton S R, Kinzler K W, Kane M F, Kolodner R D, Vogelstein B, Kunkel T A, Baylin S B. Incidence and functional consequences of hMLH1 promoter hypermethylation in colorectal carcinoma. Proc Natl Acad Sci USA 1998; 95:6870-6875.
27. Baur A S, Shaw P, Burri N, Delacretaz F, Bosman F T, Chaubert P. Frequent methylation silencing of p15(INK4b) (MTS2) and p16(INK4a) (MTS1) in B-cell and T-cell lymphomas. Blood 1999; 94:1773-1781.
28. Fang J Y, Cheng Z H, Chen Y X, Lu R, Yang L, Zhu H Y, Lu L G. Expression of Dnmt1, demethylase, MeCP2 and methylation of tumor-related genes in human gastric cancer. World J Gastroenterol 2004; 10:3394-3398.
29. Lenz G, Hutter G, Hiddemann W, Dreyling M. Promoter methylation and expression of DNA repair genes hMLH1 and MGMT in acute myeloid leukemia. Ann Hematol 2004; 83:628-633.
30. Sato M, Horio Y, Sekido Y, Minna J D, Shimokata K, Hasegawa Y. The expression of DNA methyltransferases and methyl-CpG-binding proteins is not associated with the methylation status of p14(ARF), p16(INK4a) and RASSF1A in human lung cancer cell lines. Oncogene 2002; 21:4822-4829.
31. Saito Y, Kanai Y, Sakamoto M, Saito H, Ishii H, Hirohashi S. Expression of mRNA for DNA methyltransferases and methyl-CpG-binding proteins and DNA methylation status on CpG islands and pericentromeric satellite regions during human hepatocarcinogenesis. Hepatology 2001; 33:561-568.
32. Shen L, Kondo Y, Hamilton S R, Rashid A, Issa J P. P14 methylation in human colon cancer is associated with microsatellite instability and wild-type p53. Gastroenterology 2003; 124:626-633.
33. Fang M Z, Wang Y, Ai N, Hou Z, Sun Y, Lu H, Welsh W, Yang C S. Tea polyphenol (−)-epigallocatechin-3-gallate inhibits DNA methyltransferase and reactivates methylation-silenced genes in cancer cell lines. Cancer Res 2003; 63:7563-7570.
34. Plumb J A, Strathdee G, Sludden J, Kaye S B, Brown R. Reversal of drug resistance in human tumor xenografts by 2′-deoxy-5-azacytidine-induced demethylation of the hMLH1 gene promoter. Cancer Res 2000; 60:6039-6044.
35. Lind G E, Thorstensen L, Lovig T, Meling G I, Hamelin R, Rognum T O, Esteller M, Lothe R A. A CpG island hypermethylation profile of primary colorectal carcinomas and colon cancer cell lines. Mol Cancer 2004; 3:28:28.
36. Shen L, Kondo Y, Hamilton S R, Rashid A, Issa J P. P14 methylation in human colon cancer is associated with microsatellite instability and wild-type p53. Gastroenterology 2003; 124:626-633.
37. Lee W J, Shim J Y, Zhu B T. Mechanisms for the inhibition of DNA methyltransferases by tea catechins and bioflavonoids. Mol Pharmacol 2005; 68:1018-1030.
38. Lee W J, Shim J Y, Zhu B T. Mechanisms for the inhibition of DNA methyltransferases by tea catechins and bioflavonoids. Mol Pharmacol 2005; 68:1018-1030.
39. Beltz L A, Bayer D K, Moss A L, Simet I M. Mechanisms of cancer prevention by green and black tea polyphenols. Anticancer Agents Med Chem 2006; 6:389-406.
40. Gallus S, Bosetti C, La V C. Mediterranean diet and cancer risk. Eur J Cancer Prev 2004; 13:447-452.
41. Thomasset S C, Berry D P, Garcea G, Marczylo T, Steward W P, Gescher A J. Dietary polyphenolic phytochemicals-promising cancer chemopreventive agents in humans? A review of their clinical properties. Int J Cancer 2007; 120:451-458.
42. Moos P J, Edes K, Fitzpatrick F A. Inactivation of wild-type p53 tumor suppressor by electrophilic prostaglandins. Proc Natl Acad Sci USA 2000; 97:9215-9220.
43. Graziani G, D'Argenio G, Tuccillo C, Loguercio C, Ritieni A, Morisco F, Del Vecchio B C, Fogliano V, Romano M. Apple polyphenol extracts prevent damage to human gastric epithelial cells in vitro and to rat gastric mucosa in vivo. Gut 2005; 54:193-200.
44. Zhu B T, Patel U K, Cai M X, Lee A J, Conney A H. Rapid conversion of tea catechins to monomethylated products by rat liver cytosolic catechol-O-methyltransferase. Xenobiotica 2001; 31:879-890.
45. Chuang J C, Yoo C B, Kwan J M, Li T W, Liang G, Yang A S, Jones P A. Comparison of biological effects of non-nucleoside DNA methylation inhibitors versus 5-aza-2′-deoxycytidine. Mol Cancer Ther 2005; 4:1515-1520.
46. Gaudet F, Hodgson J G, Eden A, Jackson-Grusby L, Dausman J, Gray J W, Leonhardt H, Jaenisch R. Induction of tumors in mice by genomic hypomethylation. Science 2003; 300:489-492.
47. Eden A, Gaudet F, Waghmare A, Jaenisch R. Chromosomal instability and tumors promoted by DNA hypomethylation. Science 2003; 300:455.
48. Paluszczak J, Baer-Dubowska W. Epigenetic diagnostics of cancer—the application of DNA methylation markers. J Appl Genet. 2006; 47:365-375.
49. Aparicio A, Weber J S. Review of the clinical experience with 5-azacytidine and 5-aza-2′-deoxycytidine in solid tumors. Curr Opin Investig Drugs 2002; 3:627-633.
50. Aparicio A, Eads C A, Leong L A, Laird P W, Newman E M, Synold T W, Baker S D, Zhao M, Weber J S. Phase I trial of continuous infusion 5-aza-2′-deoxycytidine. Cancer Chemother Pharmacol 2003; 51:231-239.
51. Schrump D S, Fischette M R, Nguyen D M, Zhao M, Li X, Kunst T F, Hancox A, Hong J A, Chen G A, Pishchik V, Figg W D, Murgo A J, Steinberg S M. Phase I study of decitabine-mediated gene expression in patients with cancers involving the lungs, esophagus, or pleura. Clin Cancer Res 2006; 12:5777-5785.
52. Lyko F, Brown R. DNA methyltransferase inhibitors and the development of epigenetic cancer therapies. J Natl Cancer Inst 2005; 19; 97:1498-1506.

What is claimed is:

1. A method of modulating cell proliferation comprising the steps of:
contacting one or more cells with an pharmaceutical effective amount of a polyphenolic composition comprising one or more polyphenolic compounds extracted from Annurca apple plant tissues, wherein the one or more polyphenolic compounds affects cell proliferation.

2. The method of claim 1, wherein the one or more polyphenolic compounds comprise catechins, chlorogenic acids, epicatechins or mixtures thereof.

3. The method of claim 1, wherein the apple comprises a Malus pumila L. cv Annurca apple.

4. The method of claim 1, wherein the one or more polyphenolic compounds affects cell proliferation by modifying apoptosis, induction of cell cycle arrest, decrease in cell proliferation, modulation of epigenetic changes or a combination thereof.

5. The method of claim 1, wherein the one or more cells are selected from the group consisting of carcinoma cells, colorectal cancer cells, rectal carcinoma cells, hairy cell leukemia cells, osophogeal carcinoma cells, sarcoma cells, seminoma cells, angiosarcoma cells, carcinoma cells, chordoma cells, fibrosarcoma cells, myxosarcoma cells, liposarcoma cells, chondrosarcoma cells, osteogenic sarcoma cells, endotheliosarcoma cells, lymphangiosarcoma cells, lymphangioendotheliosarcoma cells, synovioma cells, mesothelioma cells, leiomyosarcoma cells, rhabdomyosarcoma cells, pancreatic cancer cells, breast cancer cells, ovarian cancer cells, prostate cancer cells, squamous cell carcinoma cells, basal cell carcinoma cells, adenocarcinoma cells, sweat gland carcinoma cells, sebaceous gland carcinoma cells, papillary carcinoma cells, papillary adenocarcinomas cells, cystadenocarcinoma cells, medullary carcinoma cells, bronchogenic carcinoma cells, renal cell carcinoma cells, hepatoma cells, bile duct carcinoma cells, choriocarcinoma cells, embryonal carcinoma cells, cervical cancer cells, testicular tumor cells, lung carcinoma cells, small cell lung carcinoma cells, bladder carcinoma cells, epithelial hemangioblastoma cells, acoustic neuroma cells, oligodendroglioma cells, meningioma cells, melanoma cells, neuroblastoma cells, retinoblastoma cells, acute lymphocytic leukemia cells, acute myelocytic leukemia cells, promyelocytic leukemia cells, myelomonocytic leukemia cells, monocytic leukemia cells, erythroleukemia leukemia cells, chronic myelocytic leukemia cells, chronic lymphocytic leukemia cells, polycythemia vera cells, lymphoma cells, Hodgkin's disease cells, non-Hodgkin's disease cells, multiple myeloma cells, Waldenstrom's macroglobulinemia cells, Ewing's tumor cells, Wilms' tumor cells and combinations thereof.

6. A polyphenolic composition for the treatment or prevention of colorectal cancer in a subject, comprising:
a pharmaceutical carrier; and
an pharmaceutical effective amount one or more polyphenolic compounds extracted from one or more tissues of an Malus domestica [M. pumila] Annurca Tradizionale, Malus domestica [M. pumila] Annurca Rossa del Sud and combinations thereof, that the reduce the proliferation of one or more colorectal cancer cells, wherein the one or more polyphenolic compounds comprise catechins, chlorogenic acids, epicatechins or mixtures thereof.

7. The composition of claim 6, wherein the one or more polyphenolic compounds comprises between 1 mM and 20 mM of each of catechins, chlorogenic acid and epicatechin.

8. The composition of claim 6, wherein the pharmaceutical composition is in the form of an enveloped pharmaceutical comprising one or more capsules, tablets, pills, liquids, gels or mixtures thereof.

9. The composition of claim 6, wherein the polyphenolic composition is administered orally, intravenously, subcutaneously, parenterally, intraperitoneally, intraarterially, transdermally, sublingually, intramuscularly, transbuccally, intranasally, liposomally, by inhalation, by local delivery, intraadiposally, intraarticularly, intrathecally or a combination thereof.

10. The composition of claim 6, wherein the one or more polyphenolic compounds affects cell proliferation by modifying apoptosis, induction of cell cycle arrest, decrease in cell proliferation, modulation of epigenetic changes or a combination thereof.

11. A method of anticancer therapy comprising the steps of:
administering to a patient in need of anticancer therapy a pharmaceutical effective amount of a polyphenolic composition comprising one or more polyphenolic compounds extracted from one or more plant tissues, wherein the one or more polyphenolic compounds affects the proliferation of one or more cells.

12. The method of claim 11, wherein the one or more cells comprise cancer cells, precancerous cells, hyperplasia cells, prehyperplasia cells, preneoplastic cells, preneoplastic cells or mixtures thereof.

13. The method of claim 11, wherein the one or more polyphenolic compounds comprise catechins, chlorogenic acids, epicatechins or mixtures thereof.

14. The method of claim 11, wherein the one or more plant tissues comprise a tissue from Malus domestica [M. pumila] Annurca Tradizionale, Malus domestica [M. pumila] Annurca Rossa del Sud and combinations thereof.

15. The method of claim 11, wherein the polyphenolic composition is administered orally, intravenously, subcutaneously, parenterally, intraperitoneally, intraarterially, transdermally, sublingually, intramuscularly, transbuccally, intranasally, liposomally, by inhalation, by local delivery, intraadiposally, intraarticularly, intrathecally or a combination thereof.

16. The method of claim 11, further comprising administering to the patient a therapeutically effective amount of one or more anticancer treatments selected from the group consisting of radiation therapy, chemotherapy, surgery, immunotherapy, photodynamic therapy, and a combination thereof.

17. The method of claim 11, wherein the cancer is selected from the group consisting of carcinoma cells, colorectal cancer cells, rectal carcinoma cells, hairy cell leukemia cells, osophogeal carcinoma cells, sarcoma cells, seminoma cells, angiosarcoma cells, carcinoma cells, chordoma cells, fibrosarcoma cells, myxosarcoma cells, liposarcoma cells, chondrosarcoma cells, osteogenic sarcoma cells, endotheliosarcoma cells, lymphangiosarcoma cells, lymphangioendotheliosarcoma cells, synovioma cells, mesothelioma cells, leiomyosarcoma cells, rhabdomyosarcoma cells, pancreatic cancer cells, breast cancer cells, ovarian cancer cells, prostate cancer cells, squamous cell carcinoma cells, basal cell carcinoma cells, adenocarcinoma cells, sweat gland carcinoma cells, sebaceous gland carcinoma cells, papillary carcinoma cells, papillary adenocarcinomas cells, cystadenocarcinoma cells, medullary carcinoma cells, bronchogenic carcinoma cells, renal cell carcinoma cells, hepatoma cells, bile duct carcinoma cells, choriocarcinoma cells, embryonal carcinoma cells, cervical cancer cells, testicular tumor cells, lung carcinoma cells, small cell lung carcinoma cells, bladder carcinoma cells, epithelial hemangioblastoma cells, acoustic neuroma cells, oligodendroglioma cells, meningioma cells, melanoma cells, neuroblastoma cells, retinoblastoma cells, acute lymphocytic leukemia cells, acute myelocytic leukemia cells, promyelocytic leukemia cells, myelomonocytic leukemia cells, monocytic leukemia cells, erythroleukemia leukemia cells, chronic myelocytic leukemia cells, chronic lymphocytic leukemia cells, polycythemia vera cells, lymphoma cells, Hodgkin's disease cells, non-Hodgkin's disease cells, multiple myeloma cells, Waldenstrom's macroglobulinemia cells, Ewing's tumor cells, Wilms' tumor cells and combinations thereof.

18. A pharmaceutical composition for the treatment of neoplasia in a subject, comprising:
a pharmaceutical carrier; and
an pharmaceutical effective amount of one or more isolated and purified polyphenolic compounds extracted from one or more Annurca apple plant tissues, wherein the polyphenolic composition affects abnormal cell proliferation.

19. The composition of claim 18, wherein the one or more polyphenolic compounds comprise catechins, chlorogenic acids, epicatechins or mixtures thereof.

20. The composition of claim 18, wherein the one or more polyphenolic compounds comprises between 1 mM and 20 mM of each of catechins, chlorogenic acid and epicatechin.

21. The composition of claim 18, wherein the one or more plant tissues comprise a dried Annurca apple.

22. The composition of claim 18, wherein the one or more plant tissues comprise a fleshy tissue from an Annurca apple, a Malus domestica [M. pumila] Annurca Tradizionale apple, a Malus domestica [M. pumila] Annurca Rossa del Sud apple and combinations thereof.

23. The composition of claim 18, wherein the polyphenolic composition is administered orally, intravenously, subcutaneously, parenterally, intraperitoneally, intraarterially, transdermally, sublingually, intramuscularly, transbuccally, intranasally, liposomally, by inhalation, by local delivery, intraadiposally, intraarticularly, intrathecally or a combination thereof.

24. The composition of claim 18, wherein the pharmaceutical composition is in the form of an enveloped pharmaceutical comprising one or more capsules, tablets, pills, liquids, gels or mixtures thereof.

25. The composition of claim 18, wherein the one or more polyphenolic compounds reduce cell proliferation by modifying apoptosis, induction of cell cycle arrest, decrease in cell proliferation, modulation of epigenetic changes or a combination thereof.
